(19) United States
(12) Patent Application Publication
(10) Pub. No.: US 2014/0242595 A1
(43) Pub. Date: Aug. 28, 2014

(54) HEPATOCYTE PRODUCTION VIA FORWARD PROGRAMMING BY COMBINED GENETIC AND CHEMICAL ENGINEERING

(71) Applicant: Cellular Dynamics International, Inc., Madison, WI (US)

(72) Inventors: Junying YU, Madison, WI (US); Xin ZHANG, Madison, WI (US)

(73) Assignee: CELLULAR DYNAMICS INTERNATIONAL, INC., Madison, WI (US)

(21) Appl. No.: 14/186,620

(22) Filed: Feb. 21, 2014

Related U.S. Application Data
(60) Provisional application No. 61/768,301, filed on Feb. 22, 2013.

(51) Int. Cl. C12N 15/85; G01N 33/50

(52) U.S. Cl. CPC C12N 15/85; G01N 33/5067
USPC 435/6.12; 435/455; 435/29; 435/325; 435/21; 435/19; 435/7.21; 435/7.92

(57) ABSTRACT

The present invention provides methods comprising both genetic and chemical means for the production of hepatocytes from a variety of cell sources, particularly pluripotent stem cells.

The present application claims the priority benefit of U.S. provisional application No. 61/768,301, filed Feb. 22, 2013, the entire contents of which are incorporated herein by reference.

### BACKGROUND OF THE INVENTION

1. Field of the Invention

The present invention relates generally to the field of molecular biology, stem cells, and differentiated cells. More particularly, it concerns programming of somatic cells and undifferentiated cells toward specific cell lineages, particularly hepatic lineage cells.

2. Description of Related Art

In addition to their use in the transplantation therapies to treat various liver diseases, human hepatocytes are in high demand for drug toxicity screening and development due to their critical functions in the detoxification of drugs or other xenobiotics as well as endogenous substrates. Human primary hepatocytes, however, quickly lose their functions when cultured in vitro. Moreover, the drug metabolic ability of human primary hepatocytes exhibits significant differences between different individuals. The availability of an unlimited supply of patient-specific functional hepatocytes would greatly facilitate both the drug development and the eventual clinical application of hepatocyte transplantation. Therefore, there is a need for production of hepatic lineage cells in therapeutic and research use, especially, human hepatocytes.

### SUMMARY OF THE INVENTION

The present invention overcomes a major deficiency in the art in providing hepatocytes by forward programming to provide an unlimited supply of patient-specific hepatocytes. In a first embodiment there is provided a method of providing hepatocytes by genetic and chemical forward programming of a variety of cell types, including somatic cells or stem cells. Forward programming into hepatocytes may comprise increasing the expression level of certain hepatocyte programming factor genes and, in one aspect, may further comprise contacting the cells with certain small molecules to elicit forward programming of non-hepatocytes to hepatocytes.

In another embodiment, there may also be provided a method of directly programming non-hepatocytes, such as differentiation of pluripotent stem cells, into hepatocytes, comprising increasing expression of certain hepatocyte programming factor genes capable of causing forward programming to a hepatic lineage or to hepatocyte cells, therefore directly programming the cells into hepatocytes.

“Forward programming,” as used herein, refers to a process having essentially no requirement to culture cells through intermediate cellular stages using culture conditions that are adapted for each such stage and/or, optionally, having no need to add different growth factors during different time points between the starting cell source and the desired end cell product, e.g., hepatocytes, as exemplified in the upper part of FIG. 1. Forward programming may include programming of a multipotent or pluripotent cell, as opposed to a differentiated somatic cell that has lost multipotency or pluripotency, by artificially increasing the expression of one or more specific lineage-determining genes in a multipotent or pluripotent cell. For example, forward programming may describe the process of programming embryonic stem cells (ESCs) or induced pluripotent stem cells (iPSCs) to hepatocyte-like cells or other differentiated precursor or somatic cells. In certain other aspects, forward programming may refer to “trans-differentiation,” in which differentiated cells are programmed directly into another differentiated cell type without passing through an intermediate pluripotent stage.

On the other hand, the bottom part of FIG. 1 demonstrates various developmental stages present in a step-wise differentiation process and the need to add different growth factors at different times during the process, which costs more labor, time, and expenses than methods described in certain aspects of the current invention. Therefore, the methods of forward programming, in certain aspects of the present invention, are advantageous by avoiding the need to add different growth factors at different stages of programming or differentiation. For example, the medium for culturing the cells to be programmed or progeny cells thereof may be essentially free of one or more of transforming growth factors (e.g., Activin A), fibroblast growth factors (FGFs), and bone morphogenetic proteins (BMPs), which are normally required for progressive differentiation (i.e., directed differentiation as defined below) along different developmental stages.

Sources of cells suitable for hepatic forward programming may include any stem cells or non-hepatocyte somatic cells. For example, the stem cells may be pluripotent stem cells or any non-pluripotent stem cells. The pluripotent stem cells may be induced pluripotent stem cells, embryonic stem cells, or pluripotent stem cells derived by nuclear transfer or cell fusion. The stem cells may also include multipotent stem cells, oligopotent stem cells, or unipotent stem cells. The stem cells may also include fetal stem cells or adult stem cells, such as hematopoietic stem cells, mesenchymal stem cells, neural stem cells, epithelial stem cells, and skin stem cells. In certain aspects, the stem cells may be isolated from umbilical, placenta, amniotic fluid, chorion villi, blastocysts, bone marrow, adipose tissue, brain, peripheral blood, cord blood, menstrual blood, blood vessels, skeletal muscle, skin, and liver.

In other aspects, hepatocytes may be produced by transdifferentiation of non-hepatocyte somatic cells. The somatic cells for hepatic lineage programming can be any cells forming the body of an organism other than hepatocytes. In some embodiments, the somatic cells are human somatic cells, such as skin fibroblasts, adipose tissue-derived cells, and human umbilical vein endothelial cells (HUVEC). In a particular aspect, the somatic cells may be immortalized to provide an unlimited supply of cells, for example, by increasing the level of telomerase reverse transcriptase (TERT). This can be effected by increasing the transcription of TERT from the endogenous gene, or by introducing a transgene through any gene delivery method or system.

Hepatocyte programming factor genes include any genes that, alone or in combination, directly impose hepatic fate upon non-hepatocytes, especially transcription factor genes or genes that are important in hepatic differentiation or hepatic function when expressed in cells. For example, one, two, three, four, five, six, seven, eight, nine, ten, or more of the exemplary genes and isoforms or variants thereof as listed in Table 1 may be used in certain aspects of the invention. Many of these genes have different isoforms that might have similar functions and therefore are contemplated for use in certain aspects of the invention. In one embodiment of the present invention, the hepatocyte programming factor genes encoding FOXA2, GATA4, HHEX, HNF1A, MAFB, and TBX3 may be used.

In certain aspects, there is provided a method of providing hepatocytes by forward programming of pluripotent stem cells, comprising: providing the hepatocytes by culturing the pluripotent stem cells under conditions to increase the expression level of certain hepatocyte programming factor genes (e.g., by transfection of said stem cells) capable of causing forward programming of the stem cells (e.g., pluripotent stem cells) to hepatocytes, thereby causing the pluripotent stem cells to directly differentiate into hepatocytes.

The skilled artisan will understand that methods for increasing the expression of the hepatocyte programming factor genes in the cells to be programmed into hepatocytes may include any method known in the art, for example, by induction of expression of one or more expression cassettes previously introduced into the cells, or by introduction of nucleic acids, such as DNA or RNA, polypeptides, or small molecules to the cells. Increasing the expression of certain endogenous but transcriptionally repressed programming factor genes may also reverse the silencing or inhibitory effect on the expression of these programming factor genes by regulating the upstream transcription factor expression or epigenetic modulation.

In one aspect, the cells for hepatic lineage programming may comprise at least one exogenous expression cassette, wherein the expression cassette comprises the hepatocyte programming factor genes in a sufficient number to cause forward programming or transdifferentiation of non-hepatocytes to hepatocytes. The exogenous expression cassette may comprise an externally inducible transcriptional regulatory element for inducible expression of the hepatocyte programming factor genes, such as an inducible promoter comprising a tetracycline response element.

In a further aspect, one or more of the exogenous expression cassettes for hepatocyte programming may be comprised in a gene delivery system. Non-limiting examples of a gene delivery system may include a transposon system, a viral gene delivery system, an episomal gene delivery system, or a homologous recombination system. The viral gene delivery system may be an RNA-based or DNA-based viral vector. The episomal gene delivery system may be a plasmid, an Epstein-Barr virus (EBV)-based episomal vector, a yeast-based vector, an adenovirus-based vector, a simian virus 40 (SV40)-based episomal vector, a bovine papilloma virus (BPV)-based vector, or the like. The homologous recombination system may be targeting a genomic safe harbor locus, such as Rosa26 and AAVS1 loci, and may be assisted by nucleases, such as Zinc finger nuclease, TALEN, and meganucleases for improved efficiency.

In another aspect, the cells for hepatic lineage programming may be contacted with hepatocyte programming factors in an amount sufficient to cause forward programming of the stem cells to hepatocytes. The hepatocyte programming factors may comprise gene products of the hepatocyte programming factor genes. The gene products may be polypeptides or RNA transcripts of the hepatocyte programming factor genes. In a further aspect, the hepatocyte programming factors may comprise one or more protein transduction domains to facilitate their intracellular entry and/or nuclear entry. Such protein transduction domains are well known in the art, such as an HIV TAT protein transduction domain, HSV VP22 protein transduction domain, Drosophila Antennapedia homeodomain, or variants thereof.

In a certain embodiment, the stem cells comprising increased expression levels of certain hepatocyte programming factor genes are additionally contacted with a MEK inhibitor (e.g., PD0325901) and/or an ALK5 inhibitor (e.g., A 83-01) concomitantly with the induction of expression of said genes.

In a further embodiment, the stem cells are contacted with a cyclic AMP analog (e.g., 8-Br-cAMP) following the increased expression of the hepatocyte programming factor genes and/or the contacting with a MEK inhibitor and an ALK5 inhibitor.

The method may further comprise a selection or enrichment step for the hepatocytes provided from forward programming or transdifferentiation. To aid selection or enrichment, the cells for programming, such as the pluripotent stem cells or progeny cells thereof, may comprise a selectable or screenable reporter expression cassette comprising a reporter gene. The reporter expression cassette may comprise a mature hepatocyte-specific transcriptional regulatory element operably linked to a reporter gene. Non-limiting examples of hepatocyte-specific transcriptional regulatory element include a promoter of albumin, α-1-antitrypsin (AAT), cytochrome p450 3A4 (CYP3A4), apolipoprotein A-I, or apoE. The mature hepatocyte-specific transcriptional regulatory element may comprise a promoter of albumin, α1-antitrypsin, asialoglycoprotein receptor, cytokeratin 8 (CK8), cytokeratin 18 (CK18), CYP3A4, fumaryl acetoacetate hydrolase (FAH), glucose-6-phosphates, tyrosine aminotransferase, phosphoenolpyruvate carboxykinase, and tryptophan 2,3-dioxygenase.

In some aspect, the method may further comprise culturing the stem cells or progeny cells thereof as a suspension culture. In some aspects, the suspensions cultures may be maintained in spinner flasks. The spinner flasks may be operated at about 40-70 rpm. In some aspects, the suspension cultures may be maintained as static suspension cultures.

Characteristics of the hepatocytes provided in certain aspects of the invention include, but are not limited to one or more of: (i) expression of one or more hepatocyte markers, including glucose-6-phosphatase, albumin, α-1-antitrypsin (AAT), cytokeratin 8 (CK8), cytokeratin 18 (CK18), asialoglycoprotein receptor (ASGR), alcohol dehydrogenase 1, arginase Type I, cytochrome p450 3A4 (CYP3A4), liver-specific organic anion transporter (LST-1), or a combination thereof; (ii) activity of liver-specific enzymes, such as glucose-6-phosphatase or CYP3A4, production of by-products, such as bile and urea or bile secretion, or xenobiotic detoxification; (iii) hepatocyte morphological features; or (iv) in vivo liver engraftment in an immunodeficient subject.

For selection or enrichment of the hepatocytes, there may be further provided a step of identifying hepatocytes comprising expression of a hepatic reporter gene or one or more hepatocyte characteristics as described herein.

In particular aspects, the hepatocytes provided herein may be mature hepatocytes. The mature hepatocytes may be selected or enriched by using a screenable or selectable reporter expression cassette comprising a mature hepatocyte-specific transcriptional regulatory element operably linked to a reporter gene, or magnetic cell sorting using an antibody against a hepatocyte-specific cell surface antigen, such as ASGR, or by assessing characteristics specific for mature hepatocytes as known in the art. For example, mature hepatocytes can be identified by one or more of: the presence of hepatocyte growth factor receptor, albumin, α1-antitrypsin, asialoglycoprotein receptor, cytokeratin 8 (CK8), cytokeratin 18 (CK18), CYP3A4, fumaryl acetoacetate hydrolase (FAH), glucose-6-phosphates, tyrosine aminotransferase, phosphoenolpyruvate carboxykinase, and tryptophan 2,3-dioxygenase, and the absence of intracellular pancreas-associated insulin or proinsulin production. In further aspects, hepatocyte-like cells provided herein may be further forward programmed into mature hepatocytes by the artificially increased expression of genes detailed in Table 1.

For production of more mature hepatocytes, the starting cell population may be cultured in a medium comprising one or more growth factors such as Oncostain M (OSM), or further comprising hepatocyte growth factor (HGF). The culturing may be prior to, during, or after the effected expression of hepatocyte programming factors. Hepatocytes may be provided at least, about, or up to 3, 4, 5, 6, 7, 8, 9, 10, 11, 12, 13, 14, 15, 16, 17, 18, 19, 20 days (or any range derivable therein) after the increased expression or culturing in the presence or absence of growth factors.

In a further embodiment, a hepatocyte may be produced by any of the methods set forth herein. In certain aspects, there may also be provided a tissue engineered liver comprising the hepatocytes provided by the methods described herein. In another aspect, there may be provided a hepatocyte-based bio-artificial liver (BAL) comprising the hepatocytes.

In certain aspects, the invention provides a cell comprising one or more exogenous expression cassettes comprising one or more hepatocyte programming factor genes (e.g., genes in Table 1 and isoforms or variants thereof). The exogenous expression cassettes may comprise two, three, four, five, or six of the hepatocyte programming factor genes. For example, the exogenous expression cassettes may comprise the coding sequences for FOXA2, GATA4, HHEX, HNF1A, MAFB, and TBX3.

For inducible expression of the hepatocyte programming factor genes, at least one of the exogenous expression cassettes may comprise an externally inducible transcriptional regulatory element. In particular aspects, there may be provided a cell comprising one or more exogenous expression cassettes, wherein the one or more exogenous expression cassettes comprise the coding sequences for FOXA2, GATA4, HHEX, HNF1A, MAFB, and TBX3, and at least one of the exogenous expression cassettes is operably linked to an externally inducible transcriptional regulatory element.

The exogenous expression cassettes may be comprised in one or more gene delivery systems. The gene delivery system may be a transposon system; a viral gene delivery system; an episomal gene delivery system; or a homologous recombination system, such as utilizing a zinc finger nuclease, a transcription activator-like effector (TALE) nuclease, or a meganuclease, or the like. The cell may further comprise a screenable or selectable reporter expression cassette comprising a hepatocyte-specific promoter operably linked to a reporter gene. The hepatocyte-specific transcriptional regulatory element may be a promoter of albumin, α-1-antitrypsin (AAT), cytochrome p450 3A4 (CYP3A4), apolipoprotein A-I, apoE, or any other hepatocyte-specific promoter or enhancer known in the art.

In one aspect, the cell may be a stem cell or a progeny cell thereof. The stem cell may be a pluripotent stem cell or any non-pluripotent stem cell. The pluripotent stem cell may be an induced pluripotent stem cell, an embryonic stem cell, or a pluripotent stem cell derived by nuclear transfer or cell fusion. The stem cell may also be a multipotent stem cell, oligopotent stem cell, or unipotent stem cell. The stem cell may also be a fetal stem cell or an adult stem cell, for example, a hematopoietic stem cell, a mesenchymal stem cell, a neural stem cell, an epithelial stem cell, or a skin stem cell. In another aspect, the cell may be a somatic cell, either immortalized or not. The cell may also be a hepatocyte, more particularly, a mature hepatocyte or an immature hepatocyte (e.g., hepatocyte-like cell).

There may also be provided a composition comprising a cell population comprising two cell types, i.e., the cells differentiated from starting cells in response to programming culture condition changes alone and hepatocytes, and essentially free of other intermediate cell types. For example, such a cell population may have two cell types including the non-hepatic lineage cells and hepatocytes but essentially free of other cells types in the intermediate developmental stages along the hepatic differentiation process. In particular, a composition comprising a cell population consisting of non-hepatic lineage cells and hepatocytes may be provided. The non-hepatic lineage cells may be particularly epithelial cells differentiated from pluripotent stem cells, e.g., induced pluripotent stem cells. Hepatocytes may be at least, about, or up to 1%, 5%, 10%, 15%, 20%, 25%, 30%, 35%, 40%, 45%, 50%, 60%, 70%, 80%, 85%, 90%, 91%, 92%, 93%, 94%, 95%, 96%, 97%, 98%, 99%, 99.9% (or any intermediate ranges) of the cell population, or any range derivable therein.

There may be also provided a cell population comprising hepatocytes, wherein at least 20%, 25%, 30%, 35%, 40%, 45%, 50%, 60%, 70%, 80%, 85%, 90%, 91%, 92%, 93%, 94%, 95%, 96%, 97%, 98%, 99%, 99.9% (or any intermediate ranges) of the hepatocytes comprise one or more expression cassettes that comprise at least sequences encoding FOXA2, GATA4, HHEX, HNF1A, MAFB, and TBX3.

There may be provided a method of producing hepatocytes from stem cells comprising (i) transfecting the stem cells with at least one exogenous inducible expression cassette comprising at least the hepatocyte programming factor genes encoding FOXA2, GATA4, HHEX, HNF1A, MAFB, and TBX3; (ii) inducing the expression of the expression cassette for a first period of time; (iii) contacting the stem cells with a MEK inhibitor (e.g., PD0325901) and/or an ALK5 inhibitor (e.g., A 83-01) during the first period of time; and (iv) contacting the stem cells with a cyclic AMP analog (e.g., 8-Br-cAMP) for a second period of time. In certain aspects, the first and second periods of time are consecutive and non-overlapping. In some aspect, the method may further comprise culturing the stem cells or progeny cells thereof as a suspension culture. In some aspects, the suspensions cultures may be maintained in spinner flasks. The spinner flasks may be operated at about 40-70 rpm. In some aspects, the suspension cultures may be maintained as static suspension cultures.

The hepatocytes provided herein may be used in any methods and applications currently known in the art for hepatocytes. For example, a method of assessing a compound may be provided, comprising assaying a pharmacological or toxicological property of the compound on the hepatocyte or tissue engineered liver provided herein. There may also be provided a method of assessing a compound for an effect on a hepatocyte, comprising: a) contacting the hepatocyte provided herein with the compound; and b) assaying an effect of the compound on the hepatocyte.

In a further aspect, there may also be provided a method for treating a subject having or at risk of a liver dysfunction comprising administering to the subject a therapeutically effective amount of hepatocytes or a hepatocyte-containing cell population provided herein.

Embodiments discussed in the context of methods and/or compositions of the invention may be employed with respect to any other method or composition described herein. Thus, an embodiment pertaining to one method or composition may be applied to other methods and compositions of the invention as well.

As used herein the terms “encode” or “encoding” with reference to a nucleic acid are used to make the invention readily understandable by the skilled artisan however these terms may be used interchangeably with “comprise” or “comprising,” respectively.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features, and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Alternative approaches for hepatocyte differentiation from human ESC/iPSCs.

FIG. 2: The establishment of human ESC/iPSC reporter/inducible (R/I) lines for hepatocyte differentiation.

FIG. 3: Confirmation of the Tet-On inducible gene expression in human H1 ESC R/I lines. FIG. 3A: A two-vector PiggyBac stable gene expression system. Ptight: an rtTET-responsive inducible promoter; pEF: the eukaryotic elongation factor 1α promoter; hPBase: the coding region for the PiggyBac transposase with codons optimized for expression in human cells. FIG. 3B: EGFP induction in human ESC R/I lines. FIG. 3C: Flow cytometric analysis of EGFP expression in human ESC R/I lines after 4 days of induction with or without Doxycycline (1 μg/ml). Gray lines: Human ESC R/I lines without the transfection of the EGFP vector (negative control). Black lines: Human ESC R/I lines with stable PiggyBac transposon integration after 4 days of induction with or without doxycycline.

FIG. 4: Diagram of hepatocyte forward programming from human ESCs/iPSCs. Genes that are either implicated in hepatic differentiation during normal mammalian development or enriched in adult hepatocytes were cloned into the PiggyBac vector (FIG. 3) under the control of the Ptight promoter (Table 1).

FIG. 5: Transgenes and co-expression vectors for successful hepatic programming. F: FOXA2; G: GATA4; HH: HHEX; H1A: HNF1A; M: MAFB; T: TBX3; GFH: coexpression of FOXA2, GATA4 and HHEX using a bi-directional Ptight promoter where FOXA2 and HHEX were linked by a short sequence encoding the F2A peptide; H1AM: coexpression of HNF1A and MAFB using a bi-directional Ptight promoter. Both GFH and H1AM coexpression vectors have BSD as a selection marker, while all single gene expression vectors have Neo as a selection marker.

FIG. 6: Effect of MEK inhibitor PD0325901 (P) and TGFβ kinase/activin receptor like kinase (ALK5) inhibitor A 83-01 (A) on hepatic programming efficiency.

FIG. 7: Effect of doxycycline induction duration on hepatic programming. FIG. 7A: Flow cytometry analysis of ALB expression. FIG. 7B: Bright-field images of hepatic programming culture on day 12 post-plating following different days of transgene induction.

FIG. 8: Effect of cyclic AMP analog 8-Br-cAMP on hepatic programming.

FIG. 9: Effect of initial plating cell density on hepatic programming.

FIG. 10: ALB expression kinetics during hepatic programming.

FIG. 11: 3D culture facilitates hepatocyte survival and maturation. (A) The morphology of programmed hepatocytes before (Day 11) and after 4 days (Day 15) of 2D culture in HMM supplemented with insulin (0.5 μg/ml) and dexamethasone (0.1 μM). (B) Bright-field images (Days 9, 11, and 19) of 3D spheroids prepared at day 7 of programming. (C) Flow cytometry analysis of ALB expression in Day 11 3D spheroids.

### DESCRIPTION OF ILLUSTRATIVE EMBODIMENTS

The present invention overcomes several major problems with current technologies by providing methods and compositions for hepatocyte productions by forward programming using genetic and chemical means. In contrast to previous methods using step-wise differentiation protocols, certain aspects of these methods increase the level of hepatocyte programming transcription factors in non-hepatocytes to provide hepatocytes by forward programming. In addition to increasing the level of hepatocyte programming transcription factors, the non-hepatocytes may also be contacted with a MEK inhibitor and an ALK5 inhibitor to further enhance hepatocyte production. This may be further enhanced by contacting the cells undergoing forward programming with a cyclic AMP analog. Certain aspects of the present methods may be more time and cost efficient and may enable manufacture of hepatocytes for therapeutics from a renewable source, stem cells. Further embodiments and advantages of the invention are described below.

### I. DEFINITIONS

“Programming” is a process that changes a cell to form progeny of at least one new cell type, either in culture or in vivo, than it would have under the same conditions without programming. This means that after sufficient proliferation, a measurable proportion of progeny having phenotypic characteristics of the new cell type if essentially no such progeny could form before programming; alternatively, the proportion having characteristics of the new cell type is measurably more than before programming. This process includes differentiation, dedifferentiation and transdifferentiation. “Differentiation” is the process by which a less specialized cell becomes a more specialized cell type. “Dedifferentiation” is a cellular process in which a partially or terminally differentiated cell reverts to an earlier developmental stage, such as pluripotency or multipotency. “Transdifferentiation” is a process of transforming one differentiated cell type into another differentiated cell type. Under certain conditions, the proportion of progeny with characteristics of the new cell type may be at least about 1%, 5%, 25% or more in order of increasing preference.

The term “exogenous,” when used in relation to a protein, gene, nucleic acid, or polynucleotide in a cell or organism refers to a protein, gene, nucleic acid, or polynucleotide that has been introduced into the cell or organism by artificial means, or in relation a cell refers to a cell which was isolated and subsequently introduced to other cells or to an organism by artificial means. An exogenous nucleic acid may be from a different organism or cell, or it may be one or more additional copies of a nucleic acid that occurs naturally within the organism or cell. An exogenous cell may be from a different organism, or it may be from the same organism. By way of a non-limiting example, an exogenous nucleic acid is in a chromosomal location different from that of natural cells, or is otherwise flanked by a different nucleic acid sequence than that found in nature.

The term “drug” refers to a molecule including, but not limited to, small molecules, nucleic acids and proteins or combinations thereof that alter or are candidates for altering a phenotype associated with disease.

By “expression construct” or “expression cassette” is meant a nucleic acid molecule that is capable of directing transcription. An expression construct includes, at the least, one or more transcriptional control elements (such as promoters, enhancers or a structure functionally equivalent thereof) that direct gene expression in one or more desired cell types, tissues or organs. Additional elements, such as a transcription termination signal, may also be included.

A “vector” or “construct” (sometimes referred to as gene delivery system or gene transfer “vehicle”) refers to a macromolecule or complex of molecules comprising a polynucleotide to be delivered to a host cell, either in vitro or in vivo.

A “plasmid,” a common type of a vector, is an extra-chromosomal DNA molecule separate from the chromosomal DNA that is capable of replicating independently of the chromosomal DNA. In certain cases, it is circular and double-stranded.

An “origin of replication” (“ori”) or “replication origin” is a DNA sequence, e.g., in a lymphotrophic herpes virus, that when present in a plasmid in a cell is capable of maintaining linked sequences in the plasmid, and/or a site at or near where DNA synthesis initiates. An on for EBV includes FR sequences (20 imperfect copies of a 30 bp repeat), and preferably DS sequences, however, other sites in EBV bind EBNA-1, e.g., Rep* sequences can substitute for DS as an origin of replication (Kirshmaier and Sugden, 1998). Thus, a replication origin of EBV includes FR, DS or Rep* sequences or any functionally equivalent sequences through nucleic acid modifications or synthetic combination derived therefrom. For example, the present invention may also use genetically engineered replication origin of EBV, such as by insertion or mutation of individual elements, as specifically described in Lindner et al. (2008).

The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA.”

A “gene,” “polynucleotide,” “coding region,” “sequence,” “segment,” “fragment,” or “transgene” that “encodes” a particular protein is a nucleic acid molecule which is transcribed and optionally also translated into a gene product, e.g., a polypeptide, in vitro or in vivo when placed under the control of appropriate regulatory sequences. The coding region may be present in either cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

The term “control elements” refers collectively to promoter regions, polyadenylation signals, transcription termination sequences, upstream regulatory domains, origins of replication, internal ribosome entry sites (“IRES”), enhancers, splice junctions, and the like, which collectively provide for the replication, transcription, post-transcriptional processing and translation of a coding sequence in a recipient cell. Not all of these control elements need always be present so long as the selected coding sequence is capable of being replicated, transcribed and translated in an appropriate host cell.

The term “promoter” is used herein in its ordinary sense to refer to a nucleotide region comprising a DNA regulatory sequence, wherein the regulatory sequence is derived from a gene that is capable of binding RNA polymerase and initiating transcription of a downstream (3′ direction) coding sequence.

By “enhancer” is meant a nucleic acid sequence that, when positioned proximate to a promoter, confers increased transcription activity relative to the transcription activity resulting from the promoter in the absence of the enhancer domain.

By “operably linked” with reference to nucleic acid molecules is meant that two or more nucleic acid molecules (e.g., a nucleic acid molecule to be transcribed, a promoter, and an enhancer element) are connected in such a way as to permit transcription of the nucleic acid molecule. “Operably linked” with reference to peptide and/or polypeptide molecules is meant that two or more peptide and/or polypeptide molecules are connected in such a way as to yield a single polypeptide chain, i.e., a fusion polypeptide, having at least one property of each peptide and/or polypeptide component of the fusion. The fusion polypeptide is preferably chimeric, i.e., composed of heterologous molecules.

“Homology” refers to the percent of identity between two polynucleotides or two polypeptides. The correspondence between one sequence and to another can be determined by techniques known in the art. For example, homology can be determined by a direct comparison of the sequence information between two polypeptide molecules by aligning the sequence information and using readily available computer programs. Alternatively, homology can be determined by hybridization of polynucleotides under conditions that form stable duplexes between homologous regions, followed by digestion with single strand-specific nuclease(s), and size determination of the digested fragments. Two DNA, or two polypeptide, sequences are “substantially homologous” to each other when at least about 80%, preferably at least about 90%, and most preferably at least about 95% of the nucleotides, or amino acids, respectively, match over a defined length of the molecules, as determined using the methods above.

The term “cell” is herein used in its broadest sense in the art and refers to a living body that is a structural unit of tissue of a multicellular organism, is surrounded by a membrane structure that isolates it from the outside, has the capability of self replicating, and has genetic information and a mechanism for expressing it. Cells used herein may be naturally-occurring cells or artificially modified cells (e.g., fusion cells, genetically modified cells, etc.).

As used herein, the term “stem cell” refers to a cell capable of giving rising to at least one type of a more specialized cell. A stem cells has the ability to self-renew, i.e., to go through numerous cycles of cell division while maintaining the undifferentiated state, and has potency, i.e., the capacity to differentiate into specialized cell types. Typically, stem cells can regenerate an injured tissue. Stem cells herein may be, but are not limited to, embryonic stem (ES) cells, induced pluripotent stem cells, or tissue stem cells (also called tissue-specific stem cell, or somatic stem cell). Any artificially produced cell that can have the above-described abilities (e.g., fusion cells, reprogrammed cells, or the like used herein) may be a stem cell.

“Embryonic stem (ES) cells” are pluripotent stem cells derived from early embryos. An ES cell was first established in 1981, which has also been applied to production of knockout mice since 1989. In 1998, a human ES cell was established, which is currently becoming available for regenerative medicine.

Unlike ES cells, tissue stem cells have a limited differentiation potential. Tissue stem cells are present at particular locations in tissues and have an undifferentiated intracellular structure. Therefore, the pluripotency of tissue stem cells is typically low. Tissue stem cells have a higher nucleus/cytoplasm ratio and have few intracellular organelles. Most tissue stem cells have low pluripotency, a long cell cycle, and proliferative ability beyond the life of the individual. Tissue stem cells are separated into categories, based on the sites from which the cells are derived, such as the dermal system, the digestive system, the bone marrow system, the nervous system, and the like. Tissue stem cells in the dermal system include epidermal stem cells, hair follicle stem cells, and the like. Tissue stem cells in the digestive system include pancreatic (common) stem cells, liver stem cells, and the like. Tissue stem cells in the bone marrow system include hematopoietic stem cells, mesenchymal stem cells, and the like. Tissue stem cells in the nervous system include neural stem cells, retinal stem cells, and the like.

“Induced pluripotent stem cells,” commonly abbreviated as iPS cells or iPSCs, refer to a type of pluripotent stem cell artificially prepared from a non-pluripotent cell, typically an adult somatic cell, or terminally differentiated cell, such as fibroblast, a hematopoietic cell, a myocyte, a neuron, an epidermal cell, or the like, by inserting certain genes, referred to as reprogramming factors. Methods of producing and engineering iPS cells are described in U.S. patent application Ser. No. 13/546,365, which is incorporated herein in its entirety.

“Reprogramming” is a process that confers on a cell a measurably increased capacity to form progeny of at least one new cell type, either in culture or in vivo, than it would have under the same conditions without reprogramming. More specifically, reprogramming is a process that confers on a somatic cell a pluripotent potential. This means that after sufficient proliferation, a measurable proportion of progeny have phenotypic characteristics of the new cell type if essentially no such progeny could form before reprogramming; otherwise, the proportion having characteristics of the new cell type is measurably more than before reprogramming. Under certain conditions, the proportion of progeny with characteristics of the new cell type may be at least about 0.05%, 0.1%, 0.5%, 1%, 5%, 25% or more in order of increasing preference.

“Pluripotency” refers to a stem cell that has the potential to differentiate into all cells constituting one or more tissues or organs, or preferably, any of the three germ layers: endoderm (interior stomach lining, gastrointestinal tract, the lungs), mesoderm (muscle, bone, blood, urogenital), or ectoderm (epidermal tissues and nervous system). “Pluripotent stem cells” used herein refer to cells that can differentiate into cells derived from any of the three germ layers, for example, direct descendants of totipotent stem cells or induced pluripotent stem cells.

As used herein “totipotent stem cells” refers to cells that have the ability to differentiate into all cells constituting an organism, such as cells that are produced from the fusion of an egg and sperm cell. Cells produced by the first few divisions of the fertilized egg are also totipotent. These cells can differentiate into embryonic and extraembryonic cell types. Pluripotent stem cells can give rise to any fetal or adult cell type. However, alone they cannot develop into a fetal or adult animal because they lack the potential to contribute to extraembryonic tissue, such as the placenta.

In contrast, many progenitor cells are multipotent stem cells, i.e., they are capable of differentiating into a limited number of cell fates. Multipotent progenitor cells can give rise to several other cell types, but those types are limited in number. An example of a multipotent stem cell is a hematopoietic cell—a blood stem cell that can develop into several types of blood cells, but cannot develop into brain cells or other types of cells. At the end of the long series of cell divisions that form the embryo are cells that are terminally differentiated, or that are considered to be permanently committed to a specific function.

As used herein, the term “somatic cell” refers to any cell other than germ cells, such as an egg, a sperm, or the like, which does not directly transfer its DNA to the next generation. Typically, somatic cells have limited or no pluripotency. Somatic cells used herein may be naturally-occurring or genetically modified.

As used herein the term “engineered” in reference to cells refers to cells that comprise at least one genetic element exogenous to the cell that is integrated into the cell genome. In some aspects, the exogenous genetic element can be integrated at a random location in the cell genome. In other aspects, the genetic element is integrated at a specific site in the genome. For example, the genetic element may be integrated at a specific position to replace an endogenous nucleic acid sequence, such as to provide a change relative to the endogenous sequence (e.g., a change in single nucleotide position).

Cells are “substantially free” of certain undesired cell types, as used herein, when they have less that 10% of the undesired cell types, and are “essentially free” of certain cell types when they have less than 1% of the undesired cell types. However, even more desirable are cell populations wherein less than 0.5% or less than 0.1% of the total cell population comprises the undesired cell types. Thus, cell populations wherein less than 0.1% to 1% (including all intermediate percentages) of the cells of the population comprise undesirable cell types are essentially free of these cell types. A medium may be “essentially free” of certain reagents, as used herein, when there is no external addition of such agents. More preferably, these agents are absent or present at an undetectable amount.

The term “hepatocyte” as used herein is meant to include hepatocyte-like cells that exhibit some but not all characteristics of mature hepatocytes, as well as mature and fully functional hepatocytes. The cells produced by this method may be as at least as functional as the hepatocytes produced by directed differentiation to date. This technique may, as it is further improved, enable the production of completely fully functional hepatocytes, which have all characteristics of hepatocytes as determined by morphology, marker expression, and in vitro and in vivo functional assays.

The term “suspension” as used herein can refer to cell culture conditions in which cells are not attached to a solid support. Cells proliferating in suspension can be stirred while proliferating using apparatus well known to those skilled in the art.

The term “spheroid” as used herein can refer to a small aggregate of cells growing in suspension, sometimes also in combination with suspended matrix material.

### II. CELLS INVOLVED IN HEPATOCYTE PROGRAMMING

In certain embodiments of the invention, there are disclosed methods and compositions for producing hepatocytes by forward programming of cells that are not hepatocytes. There may be also provided cells that comprise exogenous expression cassettes including one or more hepatocyte programming factor genes and/or reporter expression cassettes specific for hepatocyte identification. In some embodiments, the cells may be stem cells, including but are not limited to, embryonic stem cells, fetal stem cells, or adult stem cells. In further embodiments, the cells may be any somatic cells.

A. Stem Cells

Stem cells are cells found in most, if not all, multi-cellular organisms. They are characterized by the ability to renew themselves through mitotic cell division and differentiating into a diverse range of specialized cell types. The two broad types of mammalian stem cells are: embryonic stem cells that are found in blastocysts, and adult stem cells that are found in adult tissues. In a developing embryo, stem cells can differentiate into all of the specialized embryonic tissues. In adult organisms, stem cells and progenitor cells act as a repair system for the body, replenishing specialized cells, but also maintain the normal turnover of regenerative organs, such as blood, skin or intestinal tissues.

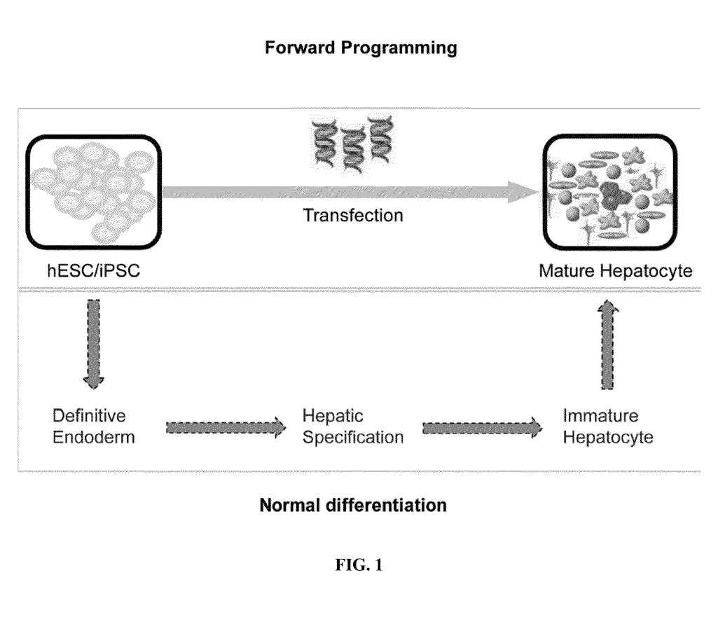

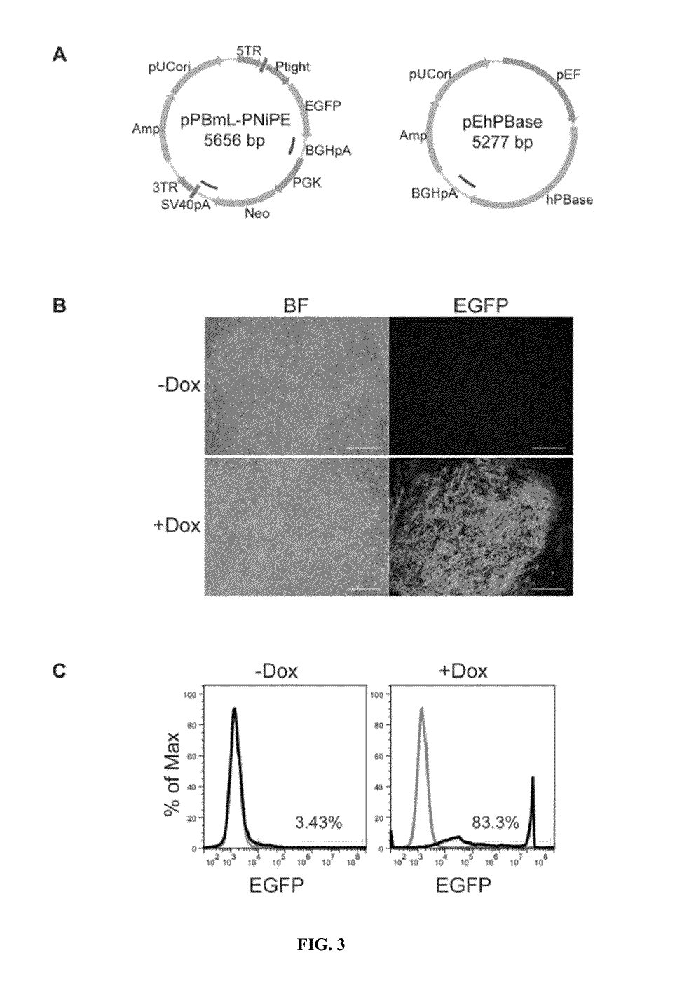

Human embryonic stem cells (ESCs) and induced pluripotent stem cells (iPSC) are capable of long-term proliferation in vitro, while retaining the potential to differentiate into all cell types of the body, including hepatocytes. Thus these cells could potentially provide an unlimited supply of patient-specific functional hepatocytes for both drug development and transplantation therapies. The differentiation of human ESC/iPSCs to hepatocytes in vitro recapitulates normal in vivo development, i.e. they undergo the following sequential developmental stages: definitive endoderm, hepatic specification, immature hepatocyte and mature hepatocyte (FIG. 1). This requires the addition of different growth factors at different stages of differentiation, and generally requires over 20 days of differentiation (FIG. 3). More importantly, the human ESC/iPSC-derived hepatocytes generally are yet to exhibit the full functional spectrum of human primary adult hepatocytes. Certain aspects of the invention provide that hepatocytes, such as hepatocyte-like cells or fully functional hepatocytes, could be induced directly from human ESC/iPSCs via expression of a combination of transcription factors important for hepatocyte differentiation/function, similar to the generation of iPSCs, bypassing most, if not all, normal developmental stages (FIG. 1). This approach could be more time and cost efficient, and generate hepatocytes with functions highly similar, if not identical, to human primary adult hepatocytes. In addition, human ESC/iPSCs, with their unlimited proliferation ability, have a unique advantage over somatic cells as the starting cell population for hepatocyte differentiation.

1. Embryonic Stem Cells

Embryonic stem cell lines (ES cell lines) are cultures of cells derived from the epiblast tissue of the inner cell mass (ICM) of a blastocyst or earlier morula stage embryos. A blastocyst is an early stage embryo, approximately four to five days old in humans and consisting of 50-150 cells. ES cells are pluripotent and give rise during development to all derivatives of the three primary germ layers: ectoderm, endoderm and mesoderm. In other words, they can develop into each of the more than 200 cell types of the adult body when given sufficient and necessary stimulation for a specific cell type. They do not contribute to the extra-embryonic membranes or the placenta.

Nearly all research to date has taken place using mouse embryonic stem cells (mES) or human embryonic stem cells (hES). Both have the essential stem cell characteristics, yet they require very different environments in order to maintain an undifferentiated state. Mouse ES cells may be grown on a layer of gelatin and require the presence of Leukemia Inhibitory Factor (LIF). Human ES cells could be grown on a feeder layer of mouse embryonic fibroblasts (MEFs) and often require the presence of basic Fibroblast Growth Factor (bFGF or FGF-2). Without optimal culture conditions or genetic manipulation (Chambers et al., 2003), embryonic stem cells will rapidly differentiate.

A human embryonic stem cell may be also defined by the presence of several transcription factors and cell surface proteins. The transcription factors Oct-4, Nanog, and Sox-2 form the core regulatory network that ensures the suppression of genes that lead to differentiation and the maintenance of pluripotency (Boyer et al., 2005). The cell surface antigens most commonly used to identify hES cells include the glycolipids SSEA3 and SSEA4 and the keratan sulfate antigens Tra-1-60 and Tra-1-81.

Methods for obtaining mouse ES cells are well known. In one method, a preimplantation blastocyst from the 129 strain of mice is treated with mouse antiserum to remove the trophoectoderm, and the inner cell mass is cultured on a feeder cell layer of chemically inactivated mouse embryonic fibroblasts in medium containing fetal calf serum. Colonies of undifferentiated ES cells that develop are subcultured on mouse embryonic fibroblast feeder layers in the presence of fetal calf serum to produce populations of ES cells. In some methods, mouse ES cells can be grown in the absence of a feeder layer by adding the cytokine leukemia inhibitory factor (LIF) to serum-containing culture medium (Smith, 2000). In other methods, mouse ES cells can be grown in serum-free medium in the presence of bone morphogenetic protein and LIF (Ying et al., 2003).

Human ES cells can be obtained from blastocysts using previously described methods (Thomson et al., 1995; Thomson et al., 1998; Thomson and Marshall, 1998; Reubinoff et al., 2000.) In one method, day-5 human blastocysts are exposed to rabbit anti-human spleen cell antiserum, then exposed to a 1:5 dilution of Guinea pig complement to lyse trophectoderm cells. After removing the lysed trophectoderm cells from the intact inner cell mass, the inner cell mass is cultured on a feeder layer of gamma-inactivated mouse embryonic fibroblasts and in the presence of fetal bovine serum. After 9 to 15 days, clumps of cells derived from the inner cell mass can be chemically (i.e. exposed to trypsin) or mechanically dissociated and replated in fresh medium containing fetal bovine serum and a feeder layer of mouse embryonic fibroblasts. Upon further proliferation, colonies having undifferentiated morphology are selected by micropipette, mechanically dissociated into clumps, and replated (see U.S. Pat. No. 6,833,269). ES-like morphology is characterized as compact colonies with apparently high nucleus to cytoplasm ratio and prominent nucleoli. Resulting ES cells can be routinely passaged by brief trypsinization or by selection of individual colonies by micropipette. In some methods, human ES cells can be grown without serum by culturing the ES cells on a feeder layer of fibroblasts in the presence of basic fibroblast growth factor (Amit et al., 2000). In other methods, human ES cells can be grown without a feeder cell layer by culturing the cells on a protein matrix such as Matrigel™ or laminin in the presence of “conditioned” medium containing basic fibroblast growth factor (Xu et al., 2001). The medium is previously conditioned by coculturing with fibroblasts.

Methods for the isolation of rhesus monkey and common marmoset ES cells are also known (Thomson, and Marshall, 1998; Thomson et al., 1995; Thomson and Odorico, 2000).

Another source of ES cells is established ES cell lines. Various mouse cell lines and human ES cell lines are known and conditions for their growth and propagation have been defined. For example, the mouse CGR8 cell line was established from the inner cell mass of mouse strain 129 embryos, and cultures of CGR8 cells can be grown in the presence of LIF without feeder layers. As a further example, human ES cell lines H1, H7, H9, H13 and H14 were established by Thompson et al. In addition, subclones H9.1 and H9.2 of the H9 line have been developed. It is anticipated that virtually any ES or stem cell line known in the art and may be used with the present invention, such as, e.g., those described in Yu and Thompson (2008), which is incorporated herein by reference.

The source of ES cells for use in connection with the present invention can be a blastocyst, cells derived from culturing the inner cell mass of a blastocyst, or cells obtained from cultures of established cell lines. Thus, as used herein, the term “ES cells” can refer to inner cell mass cells of a blastocyst, ES cells obtained from cultures of inner mass cells, and ES cells obtained from cultures of ES cell lines.

2. Induced Pluripotent Stem Cells

Induced pluripotent stem cells, commonly abbreviated iPS cells or iPSCs, are cells that have the characteristics of ES cells but are obtained by the reprogramming of differentiated, typically adult, somatic cells. Induced pluripotent stem cells are highly similar, if not identical, to embryonic stem cells in all respects that matter to pluripotency, such as in terms of expression of certain stem cell genes and proteins, chromatin methylation patterns, doubling time, embryoid body formation, teratoma formation, viable chimera formation, and potency and differentiability. iPSCs have the advantage that they are produced from cells collected from an individual thus enabling the production of cells genetically matched to the donor that can be further used to make virtually any different cell type.

Induced pluripotent stem cells have been obtained by various methods. In one method, adult human dermal fibroblasts are transfected with transcription factors Oct4, Sox2, c-Myc and Klf4 using retroviral transduction (Takahashi et al., 2007). The transfected cells are plated on SNL feeder cells (a mouse cell fibroblast cell line that produces LIF) in medium supplemented with basic fibroblast growth factor (bFGF). After approximately 25 days, colonies resembling human ES cell colonies appear in culture. The ES cell-like colonies are picked and expanded on feeder cells in the presence of bFGF.

Based on cell characteristics, cells of the ES cell-like colonies are induced pluripotent stem cells. The induced pluripotent stem cells are morphologically similar to human ES cells, and express various human ES cell markers. Also, when grown under conditions that are known to result in differentiation of human ES cells, the induced pluripotent stem cells differentiate accordingly. For example, the induced pluripotent stem cells can differentiate into cells having neuronal structures and neuronal markers. It is anticipated that virtually any iPS cells or cell lines may be used with the present invention, including, e.g., those described in Yu and Thompson (2008).

In another method, human fetal or newborn fibroblasts are transfected with four genes, Oct4, Sox2, Nanog and Lin28 using lentivirus transduction (Yu et al., 2007). At 12-20 days post infection, colonies with human ES cell morphology become visible. The colonies are picked and expanded. The induced pluripotent stem cells making up the colonies are morphologically similar to human ES cells, express various human ES cell markers, and form teratomas having neural tissue, cartilage and gut epithelium after injection into mice.

Methods of preparing induced pluripotent stem cells from mice are also known (Takahashi and Yamanaka, 2006). Induction of iPS cells typically require the expression of or exposure to at least one member from Sox family and at least one member from Oct family. Sox and Oct are thought to be central to the transcriptional regulatory hierarchy that specifies ES cell identity. For example, Sox may be Sox-1, Sox-2, Sox-3, Sox-15, or Sox-18; Oct may be Oct-4. Additional factors may increase the reprogramming efficiency, like Nanog, Lin28, Klf4, or c-Myc; specific sets of reprogramming factors may be a set comprising Sox-2, Oct-4, Nanog and, optionally, Lin-28; or comprising Sox-2, Oct4, Klf and, optionally, c-Myc.

iPS cells, like ES cells, have characteristic antigens that can be identified or confirmed by immunohistochemistry or flow cytometry, using antibodies for SSEA-1, SSEA-3 and SSEA-4 (Developmental Studies Hybridoma Bank, National Institute of Child Health and Human Development, Bethesda Md.), and TRA-1-60 and TRA-1-81 (Andrews et al., 1987). Pluripotency of embryonic stem cells can be confirmed by injecting approximately 0.5-10×10⁶ cells into the rear leg muscles of 8-12 week old male SCID mice. Teratomas develop that demonstrate at least one cell type of each of the three germ layers.

In certain aspects of the present invention, iPS cells are made from reprogramming somatic cells using reprogramming factors comprising an Oct family member and a Sox family member, such as Oct4 and Sox2 in combination with Klf or Nanog as described above. For example, a reprogramming vector may comprise expression cassettes encoding Sox2, Oct4, Nanog and optionally Lin-28, or expression cassettes encoding Sox2, Oct4, Klf4 and optionally C-myc, L-myc or Glis-1. The somatic cell for reprogramming may be any somatic cell that can be induced to pluripotency, such as a fibroblast, a keratinocyte, a hematopoietic cell, a mesenchymal cell, a liver cell, a stomach cell, or a 0 cell. In a certain aspect, T cells may also be used as source of somatic cells for reprogramming (see U.S. Application No. 61/184,546, incorporated herein by reference).

Reprogramming factors may be expressed from expression cassettes comprised in one or more vectors, such as an integrating vector or an episomal vector, e.g., an EBV element-based system (see U.S. Application No. 61/058,858, incorporated herein by reference; Yu et al., 2009). In a further aspect, reprogramming proteins or RNA (such as mRNA or miRNA) could be introduced directly into somatic cells by protein transduction or RNA transfection (see U.S. Application No. 61/172,079, incorporated herein by reference; Yakubov et al., 2010).

Oct-3/4 and certain members of the Sox gene family (Sox1, Sox2, Sox3, and Sox15) have been identified as crucial transcriptional regulators involved in the induction process whose absence makes induction impossible. Additional genes, however, including certain members of the Klf family (Klf1, Klf2, Klf4, and Klf5), the Myc family (C-myc, L-myc, and N-myc), Nanog, and LIN28, have been identified to increase the induction efficiency.

Oct-3/4 (Pou5f1) is one of the family of octamer (“Oct”) transcription factors, and plays a crucial role in maintaining pluripotency. The absence of Oct-3/4 in Oct-3/4+ cells, such as blastomeres and embryonic stem cells, leads to spontaneous trophoblast differentiation, and presence of Oct-3/4 thus gives rise to the pluripotency and differentiation potential of embryonic stem cells. Various other genes in the “Oct” family, including Oct-3/4's close relatives, Oct1 and Oct6, fail to elicit induction.

The Sox family of genes is associated with maintaining pluripotency similar to Oct-3/4, although it is associated with multipotent and unipotent stem cells in contrast with Oct-3/4, which is exclusively expressed in pluripotent stem cells. While Sox2 was the initial gene used for induction by Takahashi et al. (2006), Wernig et al. (2007), and Yu et al. (2007), other genes in the Sox family have been found to work as well in the induction process. Sox1 yields iPS cells with a similar efficiency as Sox2, and genes Sox3, Sox15, and Sox18 also generate iPS cells, although with decreased efficiency.

Nanog is a transcription factor critically involved with self-renewal of undifferentiated embryonic stem cells. In humans, this protein is encoded by the NANOG gene. Nanog is a gene expressed in embryonic stem cells (ESCs) and is thought to be a key factor in maintaining pluripotency. NANOG is thought to function in concert with other factors such as Oct4 (POU5F1) and Sox2 to establish ESC identity.

LIN28 is an mRNA binding protein expressed in embryonic stem cells and embryonic carcinoma cells associated with differentiation and proliferation. Yu et al. (2007) demonstrated it is a factor in iPS generation, although it is not essential.

Klf4 of the Klf family of genes was initially identified by Takahashi et al. (2006) and confirmed by Wernig et al. (2007) as a factor for the generation of mouse iPS cells and was demonstrated by Takahashi et al. (2007) as a factor for generation of human iPS cells. However, Yu et al. (2007) reported that Klf4 was not essential for generation of human iPS cells. Klf2 and Klf4 were found to be factors capable of generating iPS cells, and related genes Klf1 and Klf5 did as well, although with reduced efficiency.

The Myc family of genes are proto-oncogenes implicated in cancer. Takahashi et al. (2006) and Wernig et al. (2007) demonstrated that C-myc is a factor implicated in the generation of mouse iPS cells and Yamanaka et al. demonstrated it was a factor implicated in the generation of human iPS cells. However, Yu et al. (2007) and Takahashi et al. (2007) reported that c-myc was unnecessary for generation of human iPS cells. Usage of the “myc” family of genes in induction of iPS cells is troubling for the eventuality of iPS cells as clinical therapies, as 25% of mice transplanted with c-myc-induced iPS cells developed lethal teratomas. N-myc and L-myc have been identified to induce pluripotency instead of C-myc with similar efficiency. In certain aspects, Myc mutants, variants, homologs, or derivatives may be used, such as mutants that have reduced transformation of cells. Examples include LMYC (NM_{—}001033081), MYC with 41 amino acids deleted at the N-terminus (dN2MYC), or MYC with mutation at amino acid position 136 (e.g., W136E).

3. Embryonic Stem Cells Derived by Somatic Cell Nuclear Transfer

Pluripotent stem cells can be prepared by means of somatic cell nuclear transfer, in which a donor nucleus is transferred into a spindle-free oocyte. Stem cells produced by nuclear transfer are genetically identical to the donor nuclei. In one method, donor fibroblast nuclei from skin fibroblasts of a rhesus macaque are introduced into the cytoplasm of spindle-free, mature metaphase II rhesus macaque ooctyes by electrofusion (Byrne et al., 2007). The fused oocytes are activated by exposure to ionomycin, then incubated until the blastocyst stage. The inner cell mass of selected blastocysts are then cultured to produce embryonic stem cell lines. The embryonic stem cell lines show normal ES cell morphology, express various ES cell markers, and differentiate into multiple cell types both in vitro and in vivo. As used herein, the term “ES cells” refers to embryonic stem cells derived from embryos containing fertilized nuclei. ES cells are distinguished from embryonic stem cells produced by nuclear transfer, which are referred to as “embryonic stem cells derived by somatic cell nuclear transfer.”

4. Other Stem Cells

Fetal stem cells are cells with self-renewal capability and pluripotent differentiation potential. They can be isolated and expanded from fetal cytotrophoblast cells (European Patent EP0412700) and chorionic villi, amniotic fluid and the placenta (WO/2003/042405). These are hereby incorporated by reference in their entirety. Cell surface markers of fetal stem cells include CD117/c-kit⁺, SSEA3⁺, SSEA4⁺ and SSEA1⁻.

Somatic stem cells have been identified in most organ tissues. The best characterized is the hematopoietic stem cell. This is a mesoderm-derived cell that has been purified based on cell surface markers and functional characteristics. The hematopoietic stem cell, isolated from bone marrow, blood, cord blood, fetal liver and yolk sac, is the progenitor cell that reinitiates hematopoiesis for the life of a recipient and generates multiple hematopoietic lineages (see U.S. Pat. Nos. 5,635,387; 5,460,964; 5,677,136; 5,750,397; 5,759,793; 5,681,599; 5,716,827; Hill et al., 1996). These are hereby incorporated by reference in their entirety. When transplanted into lethally irradiated animals or humans, hematopoietic stem cells can repopulate the erythroid, neutrophil-macrophage, megakaryocyte and lymphoid hematopoietic cell pool. In vitro, hematopoietic stem cells can be induced to undergo at least some self-renewing cell divisions and can be induced to differentiate to the same lineages as is seen in vivo. Therefore, this cell fulfills the criteria of a stem cell.

The next best characterized is the mesenchymal stem cells (MSC), originally derived from the embryonic mesoderm and isolated from adult bone marrow, can differentiate to form muscle, bone, cartilage, fat, marrow stroma, and tendon. During embryogenesis, the mesoderm develops into limb-bud mesoderm, tissue that generates bone, cartilage, fat, skeletal muscle and possibly endothelium. Mesoderm also differentiates to visceral mesoderm, which can give rise to cardiac muscle, smooth muscle, or blood islands consisting of endothelium and hematopoietic progenitor cells. Primitive mesodermal or mesenchymal stem cells, therefore, could provide a source for a number of cell and tissue types. A number of mesenchymal stem cells have been isolated (see, for example, U.S. Pat. Nos. 5,486,359; 5,827,735; 5,811,094; 5,736,396; U.S. Pat. Nos. 5,837,539; 5,837,670; 5,827,740; Jaiswal et al., 1997; Cassiede et al., 1996; Johnstone et al., 1998; Yoo et al., 1998; Gronthos, 1994; Makino et al., 1999). These are hereby incorporated by reference in their entirety. Of the many mesenchymal stem cells that have been described, all have demonstrated limited differentiation to form only those differentiated cells generally considered to be of mesenchymal origin. To date, the most multipotent mesenchymal stem cell expresses the SH2⁺ SH4⁺ CD29⁺ CD44⁺ CD71⁺ CD90⁺ CD106⁺ CD120a⁺ CD124⁺ CD14⁻ CD34⁻ CD45⁻ phenotype.

Other stem cells have been identified, including gastrointestinal stem cells, epidermal stem cells, neural and hepatic stem cells, also termed oval cells (Potten, 1998; Watt, 1997; Alison et al, 1998).

In some embodiments, the stem cells useful for the method described herein include but are not limited to embryonic stem cells, induced pluripotent stem cells, mesenchymal stem cells, bone-marrow derived stem cells, hematopoietic stem cells, chondrocyte progenitor cells, epidermal stem cells, gastrointestinal stem cells, neural stem cells, hepatic stem cells adipose-derived mesenchymal stem cells, pancreatic progenitor cells, hair follicular stem cells, endothelial progenitor cells and smooth muscle progenitor cells.

In some embodiments, the stem cells used for the method described herein is isolated from umbilical cord, placenta, amniotic fluid, chorion villi, blastocysts, bone marrow, adipose tissue, brain, peripheral blood, the gastrointestinal tract, cord blood, blood vessels, skeletal muscle, skin, liver and menstrual blood. Stem cells prepared in the menstrual blood are called endometrial regenerative cells (Medistem Inc.).

One of ordinary skill in the art can locate, isolate and expand such stem cells. The detailed procedures for the isolation of human stem cells from various sources are described in Current Protocols in Stem Cell Biology (2007) and it is hereby incorporated by reference in its entirety. Alternatively, commercial kits and isolation systems can be used. For example, the BD FACS Aria cell sorting system, BD IMag magnetic cell separation system, and BD IMag mouse hematopoietic progenitor cell enrichment set from BD Biosciences. Methods of isolating and culturing stem cells from various sources are also described in U.S. Pat. Nos. 5,486,359, 6,991,897, 7,015,037, 7,422,736, 7,410,798, 7,410,773, and 7,399,632, each of which is hereby incorporated by reference in its entirety.

B. Somatic Cells

In certain aspects of the invention, there may also be provided methods of transdifferentiation, i.e., the direct conversion of one somatic cell type into another, e.g., deriving hepatocytes from other somatic cells. Transdifferentiation may involve the use of hepatocyte programming factor genes or gene products to increase expression levels of such genes in somatic cells for production of hepatocytes.

However, the human somatic cells may be limited in supply, especially those from living donors. In certain aspects to provide an unlimited supply of starting cells for programming, somatic cells may be immortalized by introduction of immortalizing genes or proteins, such as hTERT or oncogenes. The immortalization of cells may be reversible (e.g., using removable expression cassettes) or inducible (e.g., using inducible promoters).

Somatic cells in certain aspects of the invention may be primary cells (non-immortalized cells), such as those freshly isolated from a living organism or a progeny thereof without being established or immobilized into a cell line, or may be derived from a cell line (immortalized cells). The cells may be maintained in cell culture following their isolation from a subject. In certain embodiments the cells are passaged once or more than once (e.g., between 2-5, 5-10, 10-20, 20-50, 50-100 times, or more) prior to their use in a method of the invention. In some embodiments the cells will have been passaged no more than 1, 2, 5, 10, 20, or 50 times prior to their use in a method of the invention. They may be frozen, thawed, etc.

The somatic cells used or described herein may be native somatic cells, or engineered somatic cells, i.e., somatic cells that have been genetically altered. Somatic cells of the present invention are typically mammalian cells, such as, for example, human cells, primate cells or mouse cells. They may be obtained by well-known methods and can be obtained from any organ or tissue containing live somatic cells, e.g., blood, bone marrow, skin, lung, pancreas, liver, stomach, intestine, heart, reproductive organs, bladder, kidney, urethra and other urinary organs, etc.

Mammalian somatic cells useful in the present invention include, but are not limited to, Sertoli cells, endothelial cells, granulosa epithelial cells, neurons, pancreatic islet cells, epidermal cells, epithelial cells, hepatocytes, hair follicle cells, keratinocytes, hematopoietic cells, melanocytes, chondrocytes, lymphocytes (B and T lymphocytes), erythrocytes, macrophages, monocytes, mononuclear cells, cardiac muscle cells, and other muscle cells, etc.

In some embodiments cells are selected based on their expression of an endogenous marker known to be expressed only or primarily in a desired cell type. For example, vimentin is a fibroblast marker. Other useful markers include various keratins, cell adhesion molecules, such as cadherins, fibronectin, CD molecules, etc. The population of somatic cells may have an average cell cycle time of between 18 and 96 hours, e.g., between 24-48 hours, between 48-72 hours, etc. In some embodiments, at least 90%, 95%, 98%, 99%, or more of the cells would be expected to divide within a predetermined time such as 24, 48, 72, or 96 hours.

Methods described herein may be used to program one or more somatic cells, e.g., colonies or populations of somatic cells into hepatocytes. In some embodiments a population of cells of the present invention is substantially uniform in that at least 90% of the cells display a phenotype or characteristic of interest. In some embodiments at least 95%, 96%, 97%, 98%, 99%, 99.5%, 99.8%, 99.9%, 99.95% or more of the cells display a phenotype or characteristic of interest. In certain embodiments of the invention the somatic cells have the capacity to divide, i.e., the somatic cells are not post-mitotic.

Somatic cells may be partially or completely differentiated. Differentiation is the process by which a less specialized cell becomes a more specialized cell type. Cell differentiation can involve changes in the size, shape, polarity, metabolic activity, gene expression and/or responsiveness to signals of the cell. For example, hematopoietic stem cells differentiate to give rise to all the blood cell types including myeloid (monocytes and macrophages, neutrophils, basophils, eosinophils, erythrocytes, megakaryocytes/platelets, dendritic cells) and lymphoid lineages (T-cells, B-cells, NK-cells). During progression along the path of differentiation, the ultimate fate of a cell becomes more fixed. As described herein, both partially differentiated somatic cells and fully differentiated somatic cells can be programmed as described herein to produce desired cell types, such as hepatocytes.

### III. HEPATOCYTE PROGRAMMING FACTORS

Certain aspects of the invention provide hepatocyte programming factors for hepatocyte forward programming. The hepatocytes could be produced directly from other cell sources by increasing the level of hepatocyte programming factors in cells. The numerous functions of hepatocytes could be controlled at the transcriptional level by the concerted actions of a limited number of hepatocyte-enriched transcription factors. Any transcription factors important for hepatocyte differentiation or function may be used herein, like hepatocyte-enriched transcription factors, particularly the genes thereof listed in Table 1. All the isoforms and variants of the genes listed in Table 1 may be included in this invention, and non-limiting examples of accession numbers for certain isoforms or variants are provided.

A. Genetic Factors

For example, by effecting expression of a combination of transcription factors in Table 1, forward programming into hepatocytes from pluripotent stem cells may bypass most, if not all, normal developmental stages. The example shown is a combination of the following transcription factors: FOXA2, HHEX, HNF1A, GATA4, MAFB, and TBX3.

TABLE 1
A list of candidate genes for direct programming of human ESC/iPSCs to hepatocytes.

| | | Entrez | | |
|---|---|---|---|---|
| # | Symbol | Gene ID | Accession | Name |
| 1 | FOXA1 | 3169 | NM_004496 | forkhead box A1 |
| 2 | FOXA2 | 3170 | NM_021784 | forkhead box A2 isoform 1 |
| | | | NM_153675 | forkhead box A2 isoform 2 |
| 3 | FOXA3 | 3171 | NM_004497 | forkhead box A3 |
| 4 | GATA4 | 2626 | NM_002052 | GATA binding protein 4 |
| 5 | HHEX | 3087 | NM_002729 | hematopoietically expressed homeobox |
| 6 | TBX3 | 6926 | NM_005996 | T-box 3 isoform 1 |
| | | | NM_016569 | T-box 3 isoform 2 |
| 7 | HNF1A | 6927 | NM_000545 | HNF1 homeobox A |
| 8 | HNF4A | 3172 | NM_000457 | hepatocyte nuclear factor 4, alpha |
| 9 | MAFB | 9935 | NM_005461 | v-maf musculoaponeurotic fibrosarcoma oncogene |
| | | | | homolog B (avian) |
| 10 | ABLIM3 | 22885 | NM_014945 | actin binding LIM protein family, member 3 |
| 11 | AHR | 196 | NM_001621 | aryl hydrocarbon receptor |
| 12 | AR | 367 | NM_000044 | androgen receptor |
| 13 | ATF5 | 22809 | NM_012068 | activating transcription factor 5 |
| 14 | ATOH8 | 84913 | NM_032827 | atonal homolog 8 (Drosophila) |
| 15 | ESR1 | 2099 | NM_000125 | estrogen receptor 1 |
| 16 | NF1A | 4774 | NM_001134673 | nuclear factor I/A |
| 17 | NF1B | 4781 | NM_005596 | nuclear factor I/B |
| 18 | NR0B2 | 8431 | NM_021969 | nuclear receptor subfamily 0, group B, member 2 |
| 19 | NR1H4 | 9971 | NM_005123 | nuclear receptor subfamily 1, group H, member 4 |
| 20 | NR1I2 | 8856 | NM_003889 | nuclear receptor subfamily 1, group I, member 2, |
| | | | | isoform 1 |
| | | | NM_022002 | nuclear receptor subfamily 1, group I, member 2, |
| | | | | isoform 2 |
| 21 | NR1I3 | 9970 | NM_001077482 | nuclear receptor subfamily 1, group I, member 3, |
| | | | | transcript variant 1 |
| 22 | NR3C2 | 4306 | NM_000901 | nuclear receptor subfamily 3, group C, member 2 |
| 23 | NR5A2-2 | 2494 | NM_003822 | nuclear receptor subfamily 5, group A, member 2 |
| 24 | PPARA | 5465 | NM_005036 | PPARA peroxisome proliferator-activated receptor |
| | | | | alpha |
| 25 | PROX1 | 5629 | NM_002763 | prospero homeobox 1 |
| 26 | RORC | 6097 | NM_005060 | RAR-related orphan receptor C |
| 27 | SCML1 | 6322 | NM_001037540 | sex comb on midleg-like 1 (Drosophila) isoform a |
| | | | NM_006746 | sex comb on midleg-like 1 (Drosophila) isoform b |
| | | | NM_001037535 | sex comb on midleg-like 1 (Drosophila) isoform c |
| 28 | THRB | 7068 | NM_000461 | thyroid hormone receptor, beta (erythroblastic |
| | | | | leukemia viral (v-erb-a) oncogene homolog 2, |
| | | | | avian) |
| 29 | ZIC1 | 7545 | NM_003412 | Zic family member 1 (odd-paired homolog, |
| | | | | Drosophila) |

The hepatocyte-enriched transcription factors include, but are not limited to, hepatocyte nuclear factor 1-α (HNF-1α), -1β, -3α, -3β, -3γ, -4α, and -6 and members of the c/ebp family). Hepatocyte nuclear factors (HNFs) are a group of phylogenetically unrelated transcription factors that regulate the transcription of a diverse group of genes into proteins. These proteins include blood clotting factors and in addition, enzymes and transporters involved with glucose, cholesterol, and fatty acid transport and metabolism. Of these, HNF4A (also known as HNF4a or nuclear receptor 2A1 or (NR2A1)) and HNF1A (i.e., HNF1α) appear to be correlated with the differentiated phenotype of cultured hepatoma cells. HNF1A-null mice are viable, indicating that this factor is not an absolute requirement for the formation of an active hepatic parenchyma. In contrast, HNF4A-null mice die during embryogenesis. HNF4A is expressed early in development, visible by in situ hybridization in the mouse visceral endoderm at embryonic day 4.5, long before liver development. Whereas HNF4A appears to be essential in the visceral endoderm it may not be necessary for the earliest steps in the development of the fetal liver (Li et al., 2000).

HNF1A is also known as HNF1, LFB1, TCF1, and M0DY3. HNF1A is a transcription factor that is highly expressed in the liver and is involved in the regulation of the expression of several liver specific genes such as the human class I alcohol dehydrogenase. HNF1A (Genbank Accession No: NM_{—}000545.4) belongs to the homeobox gene family as it contains a homeobox DNA binding domain. A homeobox is a DNA sequence that binds DNA. The translated homeobox is a highly conserved stretch of 60 amino acid residues.

Forkhead box A2 (FOXA2) is also known as HNF3β, HNF3B, TCF3B and MGC19807. FOXA2 is a member of the forkhead class of DNA-binding proteins. The forkhead box is a sequence of 80 to 100 amino acids that form a motif that binds to DNA. This forkhead motif is also known as the winged helix due to the butterfly-like appearance of the loops in the protein structure of the domain. These hepatocyte nuclear factors are transcriptional activators for liver-specific genes, such as albumin and transthyretin, and they also interact with chromatin. Similar family members in mice have roles in the regulation of metabolism and in the differentiation of the pancreas and liver. This gene has been linked to sporadic cases of maturity-onset diabetes of the young. Transcript variants encoding different isoforms, isoform 1 and 2, have been identified for this gene (Genbank Accession Nos: NM 021784.4; FOXA2-1) and NM_{—}153675.2; FOXA2-2).

Hematopoietically-expressed homeobox protein HHEX is a protein that in humans is encoded by the HHEX gene. This gene encodes a member of the homeobox family of transcription factors, many of which are involved in developmental processes. HHEX is required for early development of the liver. A null mutation of HHEX results in a failure to form the liver bud and embryonic lethality.

T-box transcription factor TBX3 is a protein that in humans is encoded by the TBX3 gene. This gene is a member of a phylogenetically conserved family of genes that share a common DNA-binding domain, the T-box. T-box genes encode transcription factors involved in the regulation of developmental processes. This protein is a transcriptional repressor and is thought to play a role in the anterior/posterior axis of the tetrapod forelimb. Mutations in this gene cause ulnar-mammary syndrome, affecting limb, apocrine gland, tooth, hair, and genital development. Alternative splicing of this gene results in three transcript variants encoding different isoforms.

The Gata4 gene encodes a member of the GATA family of zinc finger transcription factors. Members of this family recognize the GATA motif, which is present in the promoters of many genes. GATA4 protein is thought to regulate genes involved in embryogenesis and in myocardial differentiation and function. Mutations in this gene have been associated with cardiac septal defects as well as reproductive defects.

The MafB gene encodes the transcription factor MAFB, which is also known as V-maf musculoaponeurotic fibrosarcoma oncogene homolog B. MAFB is a basic leucine zipper (bZIP) transcription factor that plays a role in the regulation of lineage-specific hematopoiesis by repressing ETS1-mediated transcription of erythroid-specific genes in myeloid cells. MAFB activates the insulin and glucagon promoters.

B. Chemical Factors

In certain aspects of the invention, during at least part of the reprogramming process, the cell may be maintained in the presence of one or more signaling inhibitors that inhibit a signal transducer involved in a signaling cascade, e.g., in the presence of a MEK inhibitor, a TGF-β receptor inhibitor, both a MEK inhibitor and a TGF-β receptor inhibitor, or inhibitor of other signal transducers within these same pathways.

Such a signaling inhibitor, e.g., a MEK inhibitor or a TGF-β receptor inhibitor, may be used at an effective concentration of at least or about 0.02, 0.05, 0.1, 0.2, 0.5, 1, 2, 3, 4, 5, 10, 15, 20, 25, 30, 35, 40, 45, 50, 100, 150, 200, 500 to about 1000 μM, or any range derivable therein.

2. MEK Inhibitors

MEK1 and MEK2 are dual-function serine/threonine and tyrosine protein kinases and are also known as MAP kinase kinases. Selective MEK inhibitors inhibit MEK1 and MEK2 without substantial inhibition of other enzymes. A MEK inhibitor is a compound that shows MEK inhibition when tested in the assays title “Enzyme Assays” in U.S. Pat. No. 5,525,625, which is herein incorporated by reference. A MEK inhibitor may be an ATP-competitive MEK inhibitor, a non-ATP competitive MEK inhibitor, or an ATP-uncompetitive MEK inhibitor. Examples of MEK inhibitors include, but are not limited to, AZD6244 (see WO2003/077914), PD-0325901 (Pfizer), PD-184352 (Pfizer), XL-518 (Exelixis), AR-119 (Ardea Biosciences, Valeant Pharmaceuticals), AS-7001173 (Merck Serono), AS-701255 (Merck Serono), 360770-54-3 (Wyeth), and GSK-1120212 (GlaxoSmithKline). In particular, PD184352 and PD0325901 have been found to have a high degree of specificity and potency when compared to other known MEK inhibitors (Bain et al., 2007). Other MEK inhibitors and classes of MEK inhibitors are described in Zhang et al. (2000).

3. ALK5 Inhibitors

TGF-β cytokines signal through a family of single transmembrane serine/threonine kinase receptors. These receptors can be divided in two classes, the type I or activin-like kinase (ALK) receptors and type II receptors. The ALK receptors are distinguished from the Type II receptors in that the ALK receptors (a) lack the serine/threonine rich intracellular tail, (b) possess serine/threonine kinase domains that are very homologous between Type I receptors, and (c) share a common sequence motif called the GS domain, consisting of a region rich in glycine and serine residues. The GS domain is at the amino terminal end of the intracellular kinase domain and is believed to be critical for activation by the Type II receptor. Several studies have shown that TGF-β signaling requires both the ALK (Type I) and Type II receptors. Specifically, the Type II receptor phosphorylates the GS domain of the Type I receptor for TGF-β ALK5, in the presence of TGF-β. Then ALK5, in turn, phosphorylates the cytoplasmic proteins smad2 and smad3 at two carboxy terminal serines.

Various ALK5 receptor inhibitors have been described. See, for example, U.S. Pat. Nos. 6,465,493 and 6,906,089 as well as U.S. Patent Application Publication Nos. US2003/0166633, US2004/0063745, and US2004/0039198, the contents of each of which are incorporated herein by reference. Additional ALK5 inhibitors include, but are not limited to, SB-431542 (GlaxoSmithKline), ALX-270-448 (Enzo Life Sciences), A 83-01 (Tojo et al., 2005), EW-7195 (Park et al., 2011), KI26894 (Ehata et al., 2007), LY2109761 (Eli Lilly), LY-364947 (Eli Lilly), SB-525334 (GlaxoSmithKline), SB-505124 (GlaxoSmithKline), SD-208 (Uhl et al., 2004), IN-1233 (Kim et al., 2010), and SKI2162 (SK Chemicals). Further, while an “ALK5 inhibitor” is not intended to encompass non-specific kinase inhibitors, an “ALK5 inhibitor” should be understood to encompass inhibitors that inhibit ALK4 and/or ALK7 in addition to ALK5, such as, for example, SB-431542 (see, e.g., Inman et al., 2002).

4. cAMP Analogs

Cyclic adenosine monophosphate (cAMP) is a naturally occurring compound that is present in all cells and tissues, from bacteria to humans. Examples of the cAMP derivatives useful in the present invention include, but are not limited to, N6-monoacyladenosine-3′,5′-cyclic phosphoric acid, 2′-O-monoacyladenosine-3′,5′-cyclic phosphoric acid, N6,2′-O-diacyladenosine-3′,5′-cyclic phosphoric acid or their 8-mercapto, 8-lower alkylthio, 8-benzylthio, 8-amino, 8-hydroxy, 8-chloro or 8-bromo substitution product (preferably 8-bromoadenosine 3′,5′-cyclic monophosphate), 8-benzylthioadenosine-3′,5′-cyclic phosphoric acid or its N6-lower alkyl substitution product, and 8-mercaptoadenosine-3′,5′-cyclic phosphoric acid, among which particularly preferred ones are sodium N6,2′-β-dibutyryladeno sine-3′,5′-cyclicphosphate (DBcAMP), sodium 2′-O-butyryladenosine-3′,5′-cyclic phosphate, sodium N6-butyryladenosine-3′,5′-cyclic phosphate, sodium adenosine-3′,5′-cyclic phosphate, 8-benzylthio-N6-butyryladenosine-3′,5′-cyclic phosphate, and 8-benzylthioadenosine-3′,5′-cyclic phosphate.

### IV. DELIVERY OF GENES OR GENE PRODUCTS

In certain embodiments, vectors for delivery of nucleic acids encoding hepatic lineage programming or differentiation factors could be constructed to express these factors in cells. Details of components of these vectors and delivery methods are disclosed below. In addition, protein transduction compositions or methods may be also used to effect expression of the hepatocyte programming factors.

In a further aspect, the following systems and methods may also be used in delivery of reporter expression cassette for identification of desired cell types, such as hepatocytes. In particular, a hepatocyte-specific regulatory element may be used to drive expression of a reporter gene, therefore hepatocytes derived from forward programming may be characterized, selected or enriched.

A. Nucleic Acid Delivery Systems

One of skill in the art would be well equipped to construct a vector through standard recombinant techniques (see, for example, Sambrook et al., 2001 and Ausubel et al., 1996, both incorporated herein by reference). Vectors include but are not limited to, plasmids, cosmids, viruses (bacteriophage, animal viruses, and plant viruses), and artificial chromosomes (e.g., YACs), such as retroviral vectors (e.g., derived from Moloney murine leukemia virus vectors (MoMLV), MSCV, SFFV, MPSV, SNV, etc.), lentiviral vectors (e.g., derived from HIV-1, HIV-2, SIV, BIV, FIV etc.), adenoviral (Ad) vectors, including replication competent, replication deficient and gutless forms thereof, adeno-associated viral (AAV) vectors, simian virus 40 (SV-40) vectors, bovine papilloma virus vectors, Epstein-Barr virus, herpes virus vectors, vaccinia virus vectors, Harvey murine sarcoma virus vectors, murine mammary tumor virus vectors, and Rous sarcoma virus vectors.

1. Viral Vectors

In generating recombinant viral vectors, non-essential genes are typically replaced with a gene or coding sequence for a heterologous (or non-native) protein. Viral vectors are a kind of expression construct that utilizes viral sequences to introduce nucleic acid and possibly proteins into a cell. The ability of certain viruses to infect cells or enter cells via receptor-mediated endocytosis, and to integrate into host cell genome and express viral genes stably and efficiently have made them attractive candidates for the transfer of foreign nucleic acids into cells (e.g., mammalian cells). Non-limiting examples of viral vectors that may be used to deliver a nucleic acid of certain aspects of the present invention are described below.

Retroviruses have promise as gene delivery vectors due to their ability to integrate their genes into the host genome, transferring a large amount of foreign genetic material, infecting a broad spectrum of species and cell types, and of being packaged in special cell lines (Miller, 1992).

In order to construct a retroviral vector, a nucleic acid is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation, for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136).

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference.

Likewise, adeno-associated viral (AAV) vectors can be used to mediate integration of a nucleic acid molecules into a host cell genome. For example, a gut-less AAV vector can be used such that inverted terminal repeats (ITRs) of the virus flank the nucleic acid molecule for integration. If a cell is transduced with such a vector, essentially random genome integration can be achieved. On the other hand, if cells are transduced in the presence of a functional AAV Rep gene (either in the virus or expressed in trans) then site-specific integration of the sequence at the AAVS1 integration site can be accomplished.

2. Episomal Vectors

The use of plasmid- or liposome-based extra-chromosomal (i.e., episomal) vectors may be also provided in certain aspects of the invention. Such episomal vectors may include, e.g., oriP-based vectors, and/or vectors encoding a derivative of EBNA-1. These vectors may permit large fragments of DNA to be introduced to a cell and maintained extra-chromosomally, replicated once per cell cycle, partitioned to daughter cells efficiently, and elicit substantially no immune response.

In particular, EBNA-1, the only viral protein required for the replication of the oriP-based expression vector, does not elicit a cellular immune response because it has developed an efficient mechanism to bypass the processing required for presentation of its antigens on MHC class I molecules (Levitskaya et al., 1997). Further, EBNA-1 can act in trans to enhance expression of the cloned gene, inducing expression of a cloned gene up to 100-fold in some cell lines (Langle-Rouault et al., 1998; Evans et al., 1997). Finally, the manufacture of such oriP-based expression vectors is inexpensive.

The 641 amino acids (AA) of EBNA-1 have been categorized into domains associated with its varied functions by mutational and deletional analyses. Two regions, between AA40-89 and AA329-378 are capable of linking two DNA elements in cis or in trans when bound by EBNA-1, and have thus been termed Linking Region 1 and 2 (LR1, LR2). LR1 and LR2 are functionally redundant for replication; a deletion of either one yields a derivative of EBNA-1 capable of supporting DNA replication (Mackey and Sugden, 1999; Sears et al., 2004). LR1 and LR2 are rich in arginine and glycine residues, and resemble the AT-hook motifs that bind A/T rich DNA (Aravind and Landsman, 1998), (Sears et al., 2004). An in vitro analysis of LR1 and LR2 of EBNA-1 has demonstrated their ability to bind to A/T rich DNA (Sears et al., 2004). When LR1, containing one such AT-hook, was fused to the DNA-binding and dimerization domain of EBNA-1, it was found to be sufficient for DNA replication of oriP plasmids, albeit less efficiently than the wild-type EBNA-1.

In specific embodiments of the invention, a reprogramming vector will contain both oriP and an abbreviated sequence encoding a version of EBNA-1 competent to support plasmid replication and its proper maintenance during cell division. The highly repetitive sequence within the amino-terminal one-third of wild-type EBNA-1 and removal of a 25 amino-acid region that has demonstrated toxicity in various cells are dispensable for EBNA-1's trans-acting function associated with oriP (Kennedy et al., 2003). Therefore, the abbreviated form of EBNA-1, known as deltaUR1, could be used alongside oriP within this episomal vector-based system in one embodiment.

In certain aspects, a derivative of EBNA-1 that may be used in the invention is a polypeptide which, relative to a corresponding wild-type polypeptide, has a modified amino acid sequence. The modifications include the deletion, insertion or substitution of at least one amino acid residue in a region corresponding to the unique region of LR1 (residues about 40 to about 89) in EBNA-1, and may include a deletion, insertion and/or substitution of one or more amino acid residues in regions corresponding to other residues of EBNA-1, e.g., about residue 1 to about residue 40, residues about 90 to about 328 (“Gly-Gly-Ala”repeat region), residues about 329 to about 377 (LR2), residues about 379 to about 386 (NLS), residues about 451 to about 608 (DNA binding and dimerization), or residues about 609 to about 641, so long as the resulting derivative has the desired properties, e.g., dimerizes and binds DNA containing an on corresponding to oriP, localizes to the nucleus, is not cytotoxic, and activates transcription from an extra-chromosomal but does not substantially active transcription from an integrated template.

Importantly, the replication and maintenance of oriP-based episomal vector is imperfect and is lost precipitously (25% per cell division) from cells within the first two weeks of its being introduced into cells; however, those cells that retain the plasmid lose it less frequently (3% per cell division) (Leight and Sugden, 2001; Nanbo and Sugden, 2007). Once selection for cells harboring the plasmid is removed, plasmids will be lost during each cell division until all of them have been eliminated over time without leaving a footprint of its former existence within the resulting daughter cells. Certain aspects of the invention make use of this footprint-less feature of the oriP-based system as an alternative to the current viral-associated approach to deliver genes to generate iPS cells. Other extra-chromosomal vectors will also be lost during replication and propagation of host cells and could also be employed in the present invention.

Other extra-chromosomal vectors include other lymphotrophic herpes virus-based vectors. Lymphotrophic herpes virus is a herpes virus that replicates in a lymphoblast (e.g., a human B lymphoblast) and becomes a plasmid for a part of its natural life-cycle. Herpes simplex virus (HSV) is not a “lymphotrophic” herpes virus. Exemplary lymphotrophic herpes viruses include, but are not limited to EBV, Kaposi's sarcoma herpes virus (KSHV), Herpes virus saimiri (HS) and Marek's disease virus (MDV). Also other sources of episome-base vectors are contemplated, such as yeast ARS, adenovirus, SV40, or BPV.

One of skill in the art would be well equipped to construct a vector through standard recombinant techniques (see, for example, Maniatis et al., 1988 and Ausubel et al., 1994, both incorporated herein by reference).

Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide.

Such components also might include markers, such as detectable and/or selection markers that can be used to detect or select for cells that have taken up and are expressing the nucleic acid delivered by the vector. Such components can be provided as a natural feature of the vector (such as the use of certain viral vectors that have components or functionalities mediating binding and uptake), or vectors can be modified to provide such functionalities. A large variety of such vectors are known in the art and are generally available. When a vector is maintained in a host cell, the vector can either be stably replicated by the cells during mitosis as an autonomous structure, incorporated within the genome of the host cell, or maintained in the host cell's nucleus or cytoplasm.

3. Transposon-Based Systems

According to a particular embodiment, the introduction of nucleic acids may use a transposon—transposase system. The used transposon—transposase system could be the well known Sleeping Beauty, the Frog Prince transposon—transposase system (for the description of the latter see, e.g., EP1507865), or the TTAA-specific transposon PiggyBac system.

Transposons are sequences of DNA that can move around to different positions within the genome of a single cell, a process called transposition. In the process, they can cause mutations and change the amount of DNA in the genome. Transposons were also once called jumping genes, and are examples of mobile genetic elements.

There are a variety of mobile genetic elements, and they can be grouped based on their mechanism of transposition. Class I mobile genetic elements, or retrotransposons, copy themselves by first being transcribed to RNA, then reverse transcribed back to DNA by reverse transcriptase, and then being inserted at another position in the genome. Class II mobile genetic elements move directly from one position to another using a transposase to “cut and paste” them within the genome.

4. Homologous Recombination Nuclease-Based Systems

In certain aspects of the invention, nucleic acid molecules can be introduced into cells in a specific manner for genome engineering, for example, via homologous recombination. As discussed above, some approaches to express genes in cells involve the use of viral vectors or transgenes that integrate randomly in the genome. These approaches, however, have the drawback of integration occurring either at sites that are unable to effectively mediate expression from the integrated nucleic or that result in the disruption of native genes. Problems associated with random integration could be partially overcome by homologous recombination to a specific locus in the target genome, e.g., the AAVS1 or Rosa26 locus.

Homologous recombination (HR), also known as general recombination, is a type of genetic recombination used in all forms of life in which nucleotide sequences are exchanged between two similar or identical strands of DNA. The technique has been the standard method for genome engineering in mammalian cells since the mid 1980s. The process involves several steps of physical breaking and the eventual rejoining of DNA. This process is most widely used to repair potentially lethal double-strand breaks in DNA. In addition, homologous recombination produces new combinations of DNA sequences during meiosis, the process by which eukaryotes make germ cells like sperm and ova. These new combinations of DNA represent genetic variation in offspring which allow populations to evolutionarily adapt to changing environmental conditions over time. Homologous recombination is also used in horizontal gene transfer to exchange genetic material between different strains and species of bacteria and viruses. Homologous recombination is also used as a technique in molecular biology for introducing genetic changes into target organisms.

Homologous recombination (HR) is a targeted genome modification technique that has been the standard method for genome engineering in mammalian cells since the mid 1980s. The efficiency of standard HR in mammalian cells is only 10⁻⁶ to 10⁻⁹ of cells treated (Capecchi, 1990). The use of meganucleases, or homing endonucleases, such as I-SceI have been used to increase the efficiency of HR. Both natural meganucleases as well as engineered meganucleases with modified targeting specificities have been utilized to increase HR efficiency (Pingoud and Silva, 2007; Chevalier et al., 2002). Another path toward increasing the efficiency of HR has been to engineer chimeric endonucleases with programmable DNA specificity domains (Arnould et al., 2011). Zinc-finger nucleases (ZFN) are one example of such a chimeric molecule in which zinc-finger DNA binding domains are fused with the catalytic domain of a Type IIS restriction endonuclease such as FokI (as reviewed in Durai et al., 2005; WO 05/028630).

Another class of such specificity molecules includes Transcription Activator Like Effector (TALE) DNA binding domains fused to the catalytic domain of a Type IIS restriction endonuclease such as FokI (Miller et al., 2011: PCT/IB2010/000154). TALENs can be designed for site-specific genome modification at virtually any given site of interest (Cermak et al., 2011; Christian et al., 2010; Li et al., 2011; Miller et al., 2011; Weber et al., 2011; Zhang et al., 2011). The site-specific DNA binding domain is expressed as a fusion protein with a DNA cleavage enzyme such as Fok I. The DNA binding domain is a scaffold of repeating amino acids; linking each of the repeats are two variable amino acids that bind to a single nucleotide in the DNA. For example, Asn-Asn binds guanosine, Asn-Ile binds adenosine, Asn-Gly bind thymidine, and His-Asp binds Cytosine. These two amino acids are known as the Repeat Variable Diresidue or RVD. There are many different RVD's and they can be engineered into the TAL Effector/Fokl protein construct to create a specific TALEN. The RNA encoding the recombinant TALEN can then be purified and transfected into a cell for site-specific genome modification. Once the TALEN introduces the double strand DNA break, the DNA can be modified by non-homologous end joining (NHEJ) or by homologous directed repair (HDR). This allows DNA mutagenesis, deletions, or additions depending on what additional sequences are present during the DNA repair.

B. Regulatory Elements

Eukaryotic expression cassettes included in the vectors preferably contain (in a 5′-to-3′ direction) a eukaryotic transcriptional promoter operably linked to a protein-coding sequence, splice signals, including intervening sequences, and a transcriptional termination/polyadenylation sequence.

1. Promoters/Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated that the control sequences that direct transcription and/or expression of sequences within non-nuclear organelles, such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al., 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination (as per, for example, the Eukaryotic Promoter Data Base EPDB, through world wide web at epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e.g., beta actin promoter (Ng, 1989; Quitsche et al., 1989), GADPH promoter (Alexander et al., 1988, Ercolani et al., 1988), metallothionein promoter (Karin et al., 1989; Richards et al., 1984); and concatenated response element promoters, such as cyclic AMP response element promoters (cre), serum response element promoter (sre), phorbol ester promoter (TPA), and response element promoters (tre) near a minimal TATA box. It is also possible to use human growth hormone promoter sequences (e.g., the human growth hormone minimal promoter described at Genbank, accession no. X05244, nucleotide 283-341) or a mouse mammary tumor promoter (available from the ATCC, Cat. No. ATCC 45007). A specific example could be a phosphoglycerate kinase (PGK) promoter.

Tissue-specific transgene expression, especially for reporter gene expression (such as antibiotic resistant gene expression) in hepatocytes produced from forward programming, is desirable as a way to identify produced hepatocytes. To increase both specificity and activity, the use of cis-acting regulatory elements has been contemplated. For example, a hepatocyte-specific promoter may be used, such as a promoter of albumin, α-1-antitrypsin (AAT), cytochrome p450 3A4 (CYP3A4), apolipoprotein A-I, or APOE.

In certain aspects, this also concerns enhancer sequences, i.e. nucleic acid sequences that increase a promoter's activity and that have the potential to act in cis, and regardless of their orientation, even over relatively long distances (up to several kilobases away from the target promoter). However, enhancer function is not necessarily restricted to such long distances as they may also function in close proximity to a given promoter. For the liver, numerous approaches to incorporate such organ-specific regulatory sequences into retroviral, lentiviral, adenoviral and adeno-associated viral vectors or non-viral vectors (often in addition to house-keeping hepatocyte-specific cellular promoters) have been reported so far (Ferry et al., 1998; Ghosh et al., 2000; Miao et al., 2000; Follenzi et al., 2002).

Several enhancer sequences for liver-specific genes have been documented. WO2009130208 describes several liver-specific regulatory enhancer sequences. WO95/011308 describes a gene therapy vector comprising a hepatocyte-specific control region (HCR) enhancer linked to a promoter and a transgene. The human apolipoprotein E-Hepatocyte Control Region (ApoE-HCR) is a locus control region (LCR) for liver-specific expression of the apolipoprotein E (ApoE) gene. The ApoE-HCR is located in the ApoE/CI/CII locus, has a total length of 771 bp and is important in expression of the genes ApoE and ApoC-1 in the liver (Simonet et al., 1993). In WO01/098482, the combination of this specific ApoE enhancer sequence or a truncated version thereof with hepatic promoters is suggested. It was shown that vector constructs combining the (non-truncated) ApoE-HCR enhancer with a human alpha-antitrypsin (AAT) promoter were able to produce the highest level of therapeutic protein in vivo (Miao et al., 2000) and may confer sustained expression when used in conjunction with a heterologous transgene (Miao et al., 2001).

This ApoE-HCR-AAT expression cassette as used, e.g., in the pAAV-ApoHCR-AAT-FIXIA construct (VandenDriessche et al., 2007) is one of the most potent liver-specific FIX expression constructs known, and has been successfully applied in a phase 1/2 dose-escalation clinical study in humans with severe hemophilia B (Manno et al., 2006). The expression of this hFIX minigene is driven from an ApoE-HCR joined to the human AAT promoter. The 5′-flanking sequence of the human AAT gene contains multiple cis-regulatory elements, including a distal enhancer and proximal sequences, with a total length of around 1.2 kb. It was shown to be sufficient to confer tissue specificity in vivo by driving gene expression primarily in the liver and also, to a lesser extent, in other tissues known to express AAT (Shen et al., 1989). A 347 bp fragment of this 1.2 kb region in combination with the ApoE enhancer is capable of achieving long-term liver-specific gene expression in vivo (Le et al., 1997). Interestingly, this shorter promoter targets expression to the liver with a greater specificity than that reported for larger AAT promoter fragments (Yull et al., 1995).

Other chimeric liver-specific constructs have also been proposed in the literature, e.g., with the AAT promoter and the albumin or hepatitis B enhancers (Kramer et al., 2003), or the alcohol dehydrogenase 6 (ADH6) basal promoter linked to two tandem copies of the apolipoprotein E enhancer element (Gehrke et al., 2003). The authors of the latter publication stress the importance of the relatively small size (1068 bp) of this enhancer-promoter combination.

2. Initiation Signals and Internal Ribosome Binding Sites

A specific initiation signal also may be used for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap-dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

3. Origins of Replication

In order to propagate a vector in a host cell, it may contain one or more origins of replication sites (often termed “ori”), for example, a nucleic acid sequence corresponding to oriP of EBV as described above or a genetically engineered oriP with a similar or elevated function in programming, which is a specific nucleic acid sequence at which replication is initiated. OriP is the site at or near which DNA replication initiates and is composed of two cis-acting sequences approximately 1 kilobase pair apart known as the family of repeats (FR) and the dyad symmetry (DS). Alternatively, a replication origin of other extra-chromosomally replicating virus as described above or an autonomously replicating sequence (ARS) can be employed.

4. Selection and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers, such as GFP, whose basis is colorimetric analysis, are also contemplated. Alternatively, screenable enzymes as negative selection markers, such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art. One feature of the present invention includes using selection and screenable markers to select for hepatocytes after the programming factors have effected a desired programming change in those cells.

C. Nucleic Acid Delivery

Introduction of a nucleic acid, such as DNA or RNA, into cells to be programmed with the current invention may use any suitable methods for nucleic acid delivery for transformation of a cell, as described herein or as would be known to one of ordinary skill in the art. Such methods include, but are not limited to, direct delivery of DNA, such as by ex vivo transfection (Wilson et al., 1989, Nabel et al., 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); by desiccation/inhibition-mediated DNA uptake (Potrykus et al., 1985), and any combination of such methods. Through the application of techniques such as these, organelle(s), cell(s), tissue(s) or organism(s) may be stably or transiently transformed.

1. Liposome-Mediated Transfection

In a certain embodiment of the invention, a nucleic acid may be entrapped in a lipid complex, such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is a nucleic acid complexed with Lipofectamine (Gibco BRL) or Superfect (Qiagen). The amount of liposomes used may vary upon the nature of the liposome as well as the cell used, for example, about 5 to about 20 μg vector DNA per 1 to 10 million of cells may be contemplated.

Liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa, and hepatoma cells has also been demonstrated (Wong et al., 1980).

In certain embodiments of the invention, a liposome may be complexed with a hemagglutinating virus (HVJ). This has been shown to facilitate fusion with the cell membrane and promote cell entry of liposome-encapsulated DNA (Kaneda et al., 1989). In other embodiments, a liposome may be complexed or employed in conjunction with nuclear non-histone chromosomal proteins (HMG-1) (Kato et al., 1991). In yet further embodiments, a liposome may be complexed or employed in conjunction with both HVJ and HMG-1. In other embodiments, a delivery vehicle may comprise a ligand and a liposome.

2. Electroporation

In certain embodiments of the present invention, a nucleic acid is introduced into an organelle, a cell, a tissue or an organism via electroporation. Electroporation involves the exposure of a suspension of cells and DNA to a high-voltage electric discharge. Recipient cells can be made more susceptible to transformation by mechanical wounding. Also the amount of vectors used may vary upon the nature of the cells used, for example, about 5 to about 20 μg vector DNA per 1 to 10 million of cells may be contemplated.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

3. Calcium Phosphate

In other embodiments of the present invention, a nucleic acid is introduced to the cells using calcium phosphate precipitation. Human KB cells have been transfected with adenovirus 5 DNA (Graham and Van Der Eb, 1973) using this technique. Also in this manner, mouse L (A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

4. DEAE-Dextran

In another embodiment, a nucleic acid is delivered into a cell using DEAE-dextran followed by polyethylene glycol. In this manner, reporter plasmids were introduced into mouse myeloma and erythroleukemia cells (Gopal, 1985).

D. Protein Transduction

In certain aspects of the present invention, the cells to be programmed into hepatocytes may be contacted with hepatocyte programming factors comprising polypeptides of hepatocyte transcription factor genes at a sufficient amount for forward programming. Protein transduction has been used as a method for enhancing the delivery of macromolecules into cells. Protein transduction domains may be used to introduce hepatocyte programming polypeptides or functional fragments thereof directly into cells. Research by many groups has shown that a region of the TAT protein, which is derived from the HIV Tat protein, can be fused to a target protein allowing the entry of the target protein into the cell. The mechanism of TAT mediated entry is thought to be by macropinocytosis (Gump and Dowdy, 2007).

A “protein transduction domain” or “PTD” is an amino acid sequence that can cross a biological membrane, particularly a cell membrane. When attached to a heterologous polypeptide, a PTD can enhance the translocation of the heterologous polypeptide across a biological membrane. The PTD is typically covalently attached (e.g., by a peptide bond) to the heterologous DNA binding domain. For example, the PTD and the heterologous DNA binding domain can be encoded by a single nucleic acid, e.g., in a common open reading frame or in one or more exons of a common gene. An exemplary PTD can include between 10-30 amino acids and may form an amphipathic helix. Many PTDs are basic in character. For example, a basic PTD can include at least 4, 5, 6 or 8 basic residues (e.g., arginine or lysine). A PTD may be able to enhance the translocation of a polypeptide into a cell that lacks a cell wall or a cell from a particular species, e.g., a mammalian cell, such as a human, simian, murine, bovine, equine, feline, or ovine cell.

A PTD can be linked to an artificial transcription factor, for example, using a flexible linker. Flexible linkers can include one or more glycine residues to allow for free rotation. For example, the PTD can be spaced from a DNA binding domain of the transcription factor by at least 10, 20, or 50 amino acids. A PTD can be located N- or C-terminal relative to a DNA binding domain. Being located N- or C-terminal to a particular domain does not require being adjacent to that particular domain. For example, a PTD N-terminal to a DNA binding domain can be separated from the DNA binding domain by a spacer and/or other types of domains. A PTD can be chemically synthesized then conjugated chemically to separately prepared DNA binding domain with or without linker peptide. An artificial transcription factor can also include a plurality of PTDs, e.g., a plurality of different PTDs or at least two copies of one PTD.

Several proteins and small peptides have the ability to transduce or travel through biological membranes independent of classical receptor- or endocytosis-mediated pathways. Examples of these proteins include the HIV-1 TAT protein, the herpes simplex virus 1 (HSV-1) DNA-binding protein VP22, and the Drosophila Antennapedia (Antp) homeotic transcription factor. The small protein transduction domains (PTDs) from these proteins can be fused to other macromolecules, peptides or proteins to successfully transport them into a cell. Sequence alignments of the transduction domains from these proteins show a high basic amino acid content (Lys and Arg), which may facilitate interaction of these regions with negatively charged lipids in the membrane. Secondary structure analyses show no consistent structure between all three domains.

The advantages of using fusions of these transduction domains is that protein entry is rapid, concentration-dependent, and appears to work with difficult cell types.

The Tat protein from human immunodeficiency virus type I (HIV-1) has the remarkable capacity to enter cells when added exogenously (Frankel and Pabo, 1988; Mann and Frankel, 1991; Fawell et al., 1994). The TAT PTD has been shown to successfully mediate the introduction of heterologous peptides and proteins in excess of 100 kDa into mammalian cells in vitro and in vivo (Ho et al., 2001). Schwarze et al. showed that when the 120 kDa f3-galactosidase protein fused with the TAT PTD was injected into mouse intraperitoneally, the fusion proteins were found in all types of cells and tissues even including brain, which has been thought to be difficult because of the blood-brain-barrier (Schwarze et al., 1999).

The poly-arginine peptides composed of about 6-12 arginine residues also can mediate protein transduction in some cases. For additional information about poly-arginine, see, e.g., Rothbard et al. (2000); Wender et al. (2000).

For additional information about PTDs, see also U.S. Pat. No. 6,919,425; U.S. 2003/0082561; U.S. 2003/0040038; Schwarze et al. (1999); Derossi et al. (1996); Hancock et al. (1991); Buss et al. (1988); Derossi et al. (1998); Lindgren et al. (2000); Kilic et al. (2003); Asoh et al. (2002); and Tanaka et al. (2003).

In addition to PTDs, cellular uptake signals can be used. Such signals include amino acid sequences that are specifically recognized by cellular receptors or other surface proteins. Interaction between the cellular uptake signal and the cell cause internalization of the artificial transcription factor that includes the cellular uptake signal. Some PTDs may also function by interaction with cellular receptors or other surface proteins.

A number of assays are available to determine if an amino acid sequence can function as a PTD. For example, the amino acid sequence can be fused to a reporter protein, such as β-galactosidase, to form a fusion protein. This fusion protein is contacted with cultured cells. The cells are washed and then assayed for reporter activity. Another assay detects the presence of a fusion protein that includes the amino acid sequence in question and another detectable sequence, e.g., an epitope tag. This fusion protein is contacted with culture cells. The cells are washed and then analyzed by Western or immunofluorescence to detect presence of the detectable sequence in cells. Still other assays can be used to detect transcriptional regulatory activity of a fusion protein that includes the putative PTD, a DNA binding domain, and optionally an effector domain. For example, cells contacted with such fusion proteins can be assayed for the presence or level of mRNA or protein, e.g., using microarrays, mass spectroscopy, and high-throughput techniques.

### V. CELL CULTURE

Generally, cells of the present invention are cultured in a culture medium, which is a nutrient-rich buffered solution capable of sustaining cell growth. However, the starting cell and the end, reprogrammed cell generally has differing requirements for culture medium and conditions. Likewise, when simultaneously selecting cells for integration of an engineering construct, a selective drug may be added to the culture medium during specific portions of the reprogramming process. To allow for this while also allowing that reprogramming of the cell is taking place, it is usual to carry out at least an initial stage of culture, after introduction of the reprogramming factors, in the presence of medium and under culture conditions known to be suitable for growth of the starting cell. However, this initial stage may also include a selection drug, such that only cells comprising a resistance marker proliferate during this initial growth phase.

Culture media suitable for isolating, expanding, and differentiating stem cells into hepatocytes according to the method described herein include, but are not limited, to high glucose Dulbecco's Modified Eagle's Medium (DMEM), DMEM/F-15, Liebovitz L-15, RPMI 1640, Iscove's modified Dulbecco's media (IMDM), and Opti-MEM SFM (Invitrogen Inc.). Chemically Defined Medium comprises a minimum essential medium such as Iscove's Modified Dulbecco's Medium (IMDM) (Gibco), supplemented with human serum albumin, human Ex Cyte lipoprotein, transfernin, insulin, vitamins, essential and non essential amino acids, sodium pyruvate, glutamine and a mitogen is also suitable. As used herein, a mitogen refers to an agent that stimulates cell division of a cell. An agent can be a chemical, usually some form of a protein that encourages a cell to commence cell division, triggering mitosis. In one embodiment, serum-free media, such as those described in U.S. Pat. No. 5,908,782 and WO96/39487, and the “complete media” as described in U.S. Pat. No. 5,486,359 are contemplated for use with the method described herein. In some embodiments, the culture medium is supplemented with 10% Fetal Bovine Serum (FBS), human autologous serum, human AB serum or platelet rich plasma supplemented with heparin (2 U/ml).

The medium of the present invention can also contain fatty acids or lipids, amino acids (such as non-essential amino acids), vitamin(s), growth factors, cytokines, antioxidant substances, 2-mercaptoethanol, pyruvic acid, buffering agents, and inorganic salts. The concentration of 2-mercaptoethanol can be, for example, about 0.05 to 1.0 mM, and particularly about 0.1 to 0.5 mM, but the concentration is particularly not limited thereto as long as it is appropriate for culturing the stem cell(s).

A culture vessel used for culturing the stem cell(s) can include, but is particularly not limited to: flask, flask for tissue culture, dish, petri dish, dish for tissue culture, multi dish, micro plate, micro-well plate, multi plate, multi-well plate, micro slide, chamber slide, tube, tray, CellSTACK® Chambers, culture bag, and roller bottle, as long as it is capable of culturing the stem cells therein. The stem cells may be cultured in a volume of at least or about 0.2, 0.5, 1, 2, 5, 10, 20, 30, 40, 50 ml, 100 ml, 150 ml, 200 ml, 250 ml, 300 ml, 350 ml, 400 ml, 450 ml, 500 ml, 550 ml, 600 ml, 800 ml, 1000 ml, 1500 ml, or any range derivable therein, depending on the needs of the culture. In a certain embodiment, the culture vessel may be a bioreactor, which may refer to any device or system that supports a biologically active environment. The bioreactor may have a volume of at least or about 2, 4, 5, 6, 8, 10, 15, 20, 25, 50, 75, 100, 150, 200, 500 liters, 1, 2, 4, 6, 8, 10, 15 cubic meters, or any range derivable therein.

The culture vessel can be cellular adhesive or non-adhesive and selected depending on the purpose. The cellular adhesive culture vessel can be coated with any of substrates for cell adhesion such as extracellular matrix (ECM) to improve the adhesiveness of the vessel surface to the cells. The substrate for cell adhesion can be any material intended to attach stem cells or feeder cells (if used). The substrate for cell adhesion includes collagen, gelatin, poly-L-lysine, poly-D-lysine, vitronectin, laminin, fibronectin, and RetroNectin and mixtures thereof for example Matrigel™, and lysed cell membrane preparations (Klimanskaya et al., 2005).

Other culturing conditions can be appropriately defined. For example, the culturing temperature can be about 30 to 40° C., for example, at least or about 31, 32, 33, 34, 35, 36, 37, 38, 39° C. but particularly not limited to them. The CO₂ concentration can be about 1 to 10%, for example, about 2 to 5%, or any range derivable therein. The oxygen tension can be at least or about 1, 5, 8, 10, 20%, or any range derivable therein.

Pluripotent stem cells to be differentiated into hepatocytes may be cultured in a medium sufficient to maintain the pluripotency. Culturing of induced pluripotent stem (iPS) cells generated in certain aspects of this invention can use various medium and techniques developed to culture primate pluripotent stem cells, more specially, embryonic stem cells, as described in U.S. Pat. No. 7,442,548 and U.S. Pat. App. 20030211603. For example, like human embryonic stem (hES) cells, iPS cells can be maintained in 80% DMEM (Gibco #10829-018 or #11965-092), 20% defined fetal bovine serum (FBS) not heat inactivated, 1% non-essential amino acids, 1 mM L-glutamine, and 0.1 mM beta-mercaptoethanol. Alternatively, ES cells can be maintained in serum-free medium, made with 80% Knock-Out DMEM (Gibco #10829-018), 20% serum replacement (Gibco #10828-028), 1% non-essential amino acids, 1 mM L-glutamine, and 0.1 mM beta-mercaptoethanol. Just before use, human bFGF may be added to a final concentration of about 4 ng/mL (WO 99/20741).

Hepatocytes of this invention can be made by culturing pluripotent stem cells or other non-hepatocytes in a medium under conditions that increase the intracellular level of hepatocyte programming factors to be sufficient to promote programming of the cells into hepatocytes. The medium may also contain one or more hepatocyte differentiation and maturation agents, like various kinds of growth factors. However, by increasing the intracellular level of hepatocyte programming transcription factors, aspects of the present invention bypass most stages toward mature hepatocytes without the need to change the medium for each of the stages. Therefore, in view of the advantages provided by the present invention, in particular aspects, the medium for culturing cells under hepatocyte programming may be essentially free of one or more of the hepatocyte differentiation and maturation agents, or may not undergo serial change with media containing different combination of such agents.

These agents may either help induce cells to commit to a more mature phenotype—or preferentially promote survival of the mature cells—or have a combination of both these effects. Hepatocyte differentiation and maturation agents illustrated in this disclosure may include soluble growth factors (peptide hormones, cytokines, ligand-receptor complexes, and other compounds) that are capable of promoting the growth of cells of the hepatocyte lineage. Non-limiting examples of such agents include but are not limited to epidermal growth factor (EGF), insulin, TGF-α, TGF-β, fibroblast growth factor (FGF), heparin, hepatocyte growth factor (HGF), Oncostatin M (OSM), IL-1, IL-6, insulin-like growth factors I and II (IGF-I, IGF-2), heparin binding growth factor 1 (HBGF-1), and glucagon. The skilled reader will already appreciate that Oncostatin M is structurally related to Leukemia inhibitory factor (LIF), Interleukin-6 (IL-6), and ciliary neurotrophic factor (CNTF).

An additional example is n-butyrate, as described in previous patent disclosures (U.S. Pat. No. 6,458,589, U.S. Pat. No. 6,506,574; WO 01/81549). Homologs of n-butyrate can readily be identified that have a similar effect, and can be used as substitutes in the practice of this invention. Some homologs have similar structural and physicochemical properties to those of n-butyrate: acidic hydrocarbons comprising 3-10 carbon atoms, and a conjugate base selected from the group consisting of a carboxylate, a sulfonate, a phosphonate, and other proton donors. Examples include isobutyric acid, butenoic acid, propanoic acid, other short-chain fatty acids, and dimethylbutyrate. Also included are isoteric hydrocarbon sulfonates or phosphonates, such as propanesulfonic acid and propanephosphonic acid, and conjugates such as amides, saccharides, piperazine and cyclic derivatives. A further class of butyrate homologs is inhibitors of histone deacetylase. Non-limiting examples include trichostatin A, 5-azacytidine, trapoxin A, oxamflatin, FR901228, cisplatin, and MS-27-275. Another class of agents is organic solvents like DMSO. Alternatives with similar properties include but are not limited to dimethylacetamide (DMA), hexmethylene bisacetamide, and other polymethylene bisacetamides. Solvents in this class are related, in part, by the property of increasing membrane permeability of cells. Also of interest are solutes, such as nicotinamide.

The methods of the present invention, in certain aspects, may be carried out using a suspension (or 3D) culture of cells, including suspension culture on carriers (Fernandes et al., 2004) or gel/biopolymer encapsulation (U.S. Publication 2007/0116680). The term suspension culture of the cells means that the cells are cultured under non-adherent condition with respect to the culture vessel or feeder cells (if used) in a medium. The suspension culture of cells includes a dissociation culture of cells and an aggregate suspension culture of cells. The term dissociation culture of cells means that suspended cells are cultured, and the dissociation culture of cells include those of single cells or those of small cell aggregates composed of a plurality of cells (for example, about 2 to 400 cells). When the aforementioned dissociation culture is continued, the cultured, dissociated cells form a larger aggregate of cells, and thereafter an aggregate suspension culture can be performed. The aggregate suspension culture includes an embryoid culture method (see Keller et al., 1995), and a SFEB method (Watanabe et al., 2005; International Publication No. 2005/123902).

The culture vessel used for culturing cells in suspension according to the methods of some embodiments of the invention can be any tissue culture vessel with a suitable purity grade having an internal surface designed such that cells cultured therein are unable to adhere or attach to such a surface (e.g., non-tissue culture treated cells, to prevent attachment or adherence to the surface). Preferably, in order to obtain a scalable culture, culturing according to some embodiments of the invention is effected using a controlled culturing system (preferably a computer-controlled culturing system) in which culture parameters such as temperature, agitation, pH, and pO₂ is automatically performed using a suitable device. Once the culture parameters are recorded, the system is set for automatic adjustment of culture parameters as needed for promotion of cell expansion. Cells may be cultured under dynamic conditions (i.e., under conditions in which the cells are subject to constant movement while in the suspension culture) or under non-dynamic conditions (i.e., a static culture) while preserving their proliferative capacity. For non-dynamic culturing of cells, the cells can be cultured in uncoated 58 mm Petri dishes (Greiner, Frickenhausen, Germany). For dynamic culturing of cells, the cells can be cultured in spinner flasks (e.g., of 200 ml to 1000 ml, for example 250 ml; of 100 ml; or in 125 ml Erlenmeyer) which can be connected to a control unit and thus present a controlled culturing system. The culture vessel (e.g., a spinner flask, an Erlenmeyer) is shaken continuously. According to some embodiments of the invention the culture vessels are shaken at 90 rounds per minute (rpm) using a shaker. According to some embodiments of the invention the culture medium is changed daily.

Based on the source of cells and the need for expansion, the dissociated cells may be transferred individually or in small clusters to new culture containers in a splitting ratio such as at least or about 1:2, 1:4, 1:5, 1:6, 1:8, 1:10, 1:20, 1:40, 1:50, 1:100, 1:150, 1:200, or any range derivable therein. Suspension cell line split ratios may be done on volume of culture cell suspension. The passage interval may be at least or about every 1, 2, 3, 4, 5, 6, 7, 8, 9, 10, 11, 12, 13, 14, 15, 16, 17, 18, 19, 20 days or any range derivable therein. For example, the achievable split ratios for the different enzymatic passaging protocols may be 1:2 every 3-7 days, 1:3 every 4-7 days, and 1:5 to 1:10 approximately every 7 days, 1:50 to 1:100 every 7 days. When high split ratios are used, the passage interval may be extended to at least 12-14 days or any time period without cell loss due to excessive spontaneous differentiation or cell death.

### VI. HEPATOCYTE CHARACTERISTICS

Cells can be characterized according to a number of phenotypic criteria. The criteria include but are not limited to the detection or quantitation of expressed cell markers, enzymatic activity, and the characterization of morphological features and intercellular signaling. In other aspects, cells to be programmed may comprise reporter gene expression cassette comprising tissue- or cell-specific transcriptional regulatory element, like hepatocyte-specific promoters for hepatocyte identification.

Hepatocytes embodied in certain aspects of this invention have morphological features characteristic of hepatocytes in the nature, such as primary hepatocytes from organ sources. The features are readily appreciated by those skilled in evaluating such things and include any or all of the following: a polygonal cell shape, a binucleate phenotype, the presence of rough endoplasmic reticulum for synthesis of secreted protein, the presence of Golgi-endoplasmic reticulum lysosome complex for intracellular protein sorting, the presence of peroxisomes and glycogen granules, relatively abundant mitochondria, and the ability to form tight intercellular junctions resulting in creation of bile canalicular spaces. A number of these features present in a single cell are consistent with the cell being a member of the hepatocyte lineage. Unbiased determination of whether cells have morphologic features characteristic of hepatocytes can be made by coding micrographs of programming progeny cells, adult or fetal hepatocytes, and one or more negative control cells, such as a fibroblast, or RPE (Retinal pigment epithelial) cells—then evaluating the micrographs in a blinded fashion, and breaking the code to determine if the cells produced from forward programming are accurately identified.

Cells of this invention can also be characterized according to whether they express phenotypic markers characteristic of cells of the hepatocyte lineage. Non-limiting examples of cell markers useful in distinguishing hepatocytes include albumin, asialoglycoprotein receptor, α1-antitrypsin, α-fetoprotein, apoE, arginase I, apoAI, apoAII, apoB, apoCIII, apoCII, aldolase B, alcohol dehydrogenase 1, catalase, CYP3A4, glucokinase, glucose-6-phosphatase, insulin growth factors 1 and 2, IGF-1 receptor, insulin receptor, leptin, liver-specific organic anion transporter (LST-1), L-type fatty acid binding protein, phenylalanine hydroxylase, transferrin, retinol binding protein, and erythropoietin (EPO). Mature hepatocyte markers include, but are limited to, albumin, α1-antitrypsin, asialoglycoprotein receptor, cytokeratin 8 (CK8), cytokeratin 18 (CK18), CYP3A4, fumaryl acetoacetate hydrolase (FAH), glucose-6-phosphates, tyrosine aminotransferase, phosphoenolpyruvate carboxykinase, and tryptophan 2,3-dioxygenase.

Assessment of the level of expression of such markers can be determined in comparison with other cells. Positive controls for the markers of mature hepatocytes include adult hepatocytes of the species of interest, and established hepatocyte cell lines. The reader is cautioned that permanent cell lines or long-term liver cell cultures may be metabolically altered, and fail to express certain characteristics of primary hepatocytes. Negative controls include cells of a separate lineage, such as an adult fibroblast cell line, or retinal pigment epithelial (RPE) cells. Undifferentiated stem cells are positive for some of the markers listed above, but negative for markers of mature hepatocytes, as illustrated in the examples below.

Tissue-specific (e.g., hepatocyte-specific) protein and oligosaccharide determinants listed in this disclosure can be detected using any suitable immunological technique—such as flow immunocytochemistry for cell-surface markers, immunohistochemistry (for example, of fixed cells or tissue sections) for intracellular or cell-surface markers, Western blot analysis of cellular extracts, and enzyme-linked immunoassay, for cellular extracts or products secreted into the medium. Expression of an antigen by a cell is said to be “antibody-detectable” if a significantly detectable amount of antibody will bind to the antigen in a standard immunocytochemistry or flow cytometry assay, optionally after fixation of the cells, and optionally using a labeled secondary antibody or other conjugate (such as a biotin-avidin conjugate) to amplify labeling.

The expression of tissue-specific (e.g., hepatocyte-specific) markers can also be detected at the mRNA level by Northern blot analysis, dot-blot hybridization analysis, or by real-time polymerase chain reaction (PCR) using sequence-specific primers in standard amplification methods (U.S. Pat. No. 5,843,780). Sequence data for the particular markers listed in this disclosure can be obtained from public databases, such as GenBank. Expression at the mRNA level is said to be “detectable” according to one of the assays described in this disclosure if the performance of the assay on cell samples according to standard procedures in a typical controlled experiment results in clearly discernable hybridization or amplification product within a standard time window. Unless otherwise required, expression of a particular marker is indicated if the corresponding mRNA is detectable by RT-PCR. Expression of tissue-specific markers as detected at the protein or mRNA level is considered positive if the level is at least 2-fold, and preferably more than 10- or 50-fold above that of a control cell, such as an undifferentiated pluripotent stem cell, a fibroblast, or other unrelated cell type.

Cells can also be characterized according to whether they display enzymatic activity that is characteristic of cells of the hepatocyte lineage. For example, assays for glucose-6-phosphatase activity are described by Bublitz (1991); Yasmineh et al. (1992); and Ockerman (1968). Assays for alkaline phosphatase (ALP) and 5-nucleotidase (5′-Nase) in liver cells are described by Shiojiri (1981). A number of laboratories that serve the research and health care sectors provide assays for liver enzymes as a commercial service.

In other embodiments, cells of the invention are assayed for activity indicative of xenobiotic detoxification. Cytochrome p450 is a key catalytic component of the mono-oxygenase system. It constitutes a family of hemoproteins responsible for the oxidative metabolism of xenobiotics (administered drugs), and many endogenous compounds. Different cytochromes present characteristic and overlapping substrate specificity. Most of the biotransforming ability is attributable by the cytochromes designated 1A2, 2A6, 2B6, 3A4, 2C 9-11, 2D6, and 2E1 (Gomes-Lechon et al., 1997).

A number of assays are known in the art for measuring xenobiotic detoxification by cytochrome p450 enzyme activity. Detoxification by CYP3A4 is demonstrated using the P450-Glo™ CYP3A4 DMSO-tolerance assay (Luciferin-PPXE) and the P450-Glo™ CYP3A4 cell-based/biochemical assay (Luciferin-PFBE) (Promega Inc, #V8911 and #V8901). Detoxification by CYP1A1 and or CYP1B1 is demonstrated using the P450-Glo™ assay (Luciferin-CEE) (Promega Inc., #V8762). Detoxification by CYP1A2 and or CYP4A is demonstrated using the P450-Glo™ assay (Luciferin-ME) (Promega Inc., #V8772). Detoxification by CYP2C9 is demonstrated using the P450-Glo™ CYP2C9 assay (Luciferin-H) (Promega Inc., #V8791).

In another aspect, the biological function of a hepatocyte cell provided by programming is evaluated, for example, by analyzing glycogen storage. Glycogen storage is characterized by assaying Periodic Acid Schiff (PAS) functional staining for glycogen granules. The hepatocyte-like cells are first oxidized by periodic acid. The oxidative process results in the formation of aldehyde groupings through carbon-to-carbon bond cleavage. Free hydroxyl groups should be present for oxidation to take place. Oxidation is completed when it reaches the aldehyde stage. The aldehyde groups are detected by the Schiff reagent. A colorless, unstable dialdehyde compound is formed and then transformed to the colored final product by restoration of the quinoid chromophoric grouping (Thompson, 1966; Sheehan and Hrapchak, 1987). PAS staining can be performed according the protocol described on the world wide web at jhu.edu/˜iic/PDF jrotocols/LM/Glycogen Staining pdf and library.med.utah.edu/WebPath/HISTHTML/MANUALS/PAS.PDF with some modifications for an in vitro culture of hepatocyte-like cells. One of ordinary skill in the art should be able to make the appropriate modifications.

In another aspect, a hepatocyte cell produced by forward programming in certain aspects of the invention is characterized for urea production. Urea production can be assayed colorimetrically using kits from Sigma Diagnostic (Miyoshi et al., 1998) based on the biochemical reaction of urease reduction to urea and ammonia and the subsequent reaction with 2-oxoglutarate to form glutamate and NAD.

In another aspect, bile secretion is analyzed. Biliary secretion can be determined by fluorescein diacetate time lapse assay. Briefly, monolayer cultures of hepatocyte-like cells are rinsed with phosphate buffered saline (PBS) three times and incubated with serum-free hepatocyte growth media supplemented with doxycycline and fluorescein diacetate (20 μg/ml) (Sigma-Aldrich) at 37° C. for 35 minutes. The cells are washed with PBS three times and fluorescence imaging is carried out. Fluorescein diacetate is a non fluorescent precursor of fluorescein. The image is evaluated to determine that the compound had been taken up and metabolized in the hepatocyte-like cell to fluorescein. In some embodiments, the compound is secreted into intercellular clefts of the monolayer of cells. Alternatively, bile secretion is determined by a method using sodium fluorescein described by Gebhart and Wang (1982).

In yet another aspect, lipid synthesis is analyzed. Lipid synthesis in the hepatocyte-like cell can be determined by oil red O staining Oil Red O (Solvent Red 27, Sudan Red 5B, C.I. 26125, C26H24N4O) is a lysochrome (fat-soluble dye) diazo dye used for staining of neutral triglycerides and lipids on frozen sections and some lipoproteins on paraffin sections. It has the appearance of a red powder with maximum absorption at 518(359) nm. Oil Red O is one of the dyes used for Sudan staining. Similar dyes include Sudan III, Sudan IV, and Sudan Black B. The staining has to be performed on fresh samples and/or formalin fixed samples. Hepatocyte-like cells are cultured on microscope slides, rinsed in PBS three times, the slides are air dried for 30-60 minutes at room temperature, fixed in ice cold 10% formalin for 5-10 minutes, and then rinse immediately in three changes of distilled water. The slide is then placed in absolute propylene glycol for 2-5 minutes to avoid carrying water into Oil Red O and stained in pre-warmed Oil Red O solution for 8 minutes in 600° C. oven. The slide is then placed in 85% propylene glycol solution for 2-5 minutes and rinsed in two changes of distilled water. Oil red O staining can also be performed according the protocol described on the world wide web at library.med.utah.edu/WebPath/HISTHTML/MANUALS/OILRED.PDF with some modifications for an in vitro culture of hepatocyte-like cell by one of ordinary skill in the art.

In still another aspect, the cells are assayed for glycogen synthesis. Glycogen assays are well known to one of ordinary skill in the art, for example, in Passonneau and Lauderdale (1974). Alternatively, commercial glycogen assays can be used, for example, from BioVision, Inc. catalog #K646-100.

Cells of the hepatocyte lineage can also be evaluated by their ability to store glycogen. A suitable assay uses Periodic Acid Schiff (PAS) stain, which does not react with mono- and disaccharides, but stains long-chain polymers, such as glycogen and dextran. PAS reaction provides quantitative estimations of complex carbohydrates as well as soluble and membrane-bound carbohydrate compounds. Kirkeby et al. (1992) describe a quantitative PAS assay of carbohydrate compounds and detergents. van der Laarse et al. (1992) describe a microdensitometric histochemical assay for glycogen using the PAS reaction. Evidence of glycogen storage is determined if the cells are PAS-positive at a level that is at least 2-fold, and preferably more than 10-fold above that of a control cell, such as a fibroblast. The cells can also be characterized by karyotyping according to standard methods.

Assays are also available for enzymes involved in the conjugation, metabolism, or detoxification of small molecule drugs. For example, cells can be characterized by an ability to conjugate bilirubin, bile acids, and small molecule drugs, for excretion through the urinary or biliary tract. Cells are contacted with a suitable substrate, incubated for a suitable period, and then the medium is analyzed (by GCMS or other suitable technique) to determine whether a conjugation product has been formed. Drug metabolizing enzyme activities include de-ethylation, dealkylation, hydroxylation, demethylation, oxidation, glucuroconjugation, sulfoconjugation, glutathione conjugation, and N-acetyl transferase activity (Guillouzo, 1997). Assays include peenacetin de-ethylation, procainamide N-acetylation, paracetamol sulfoconjugation, and paracetamol glucuronidation (Chesne et al., 1988).

A further feature of certain cell populations of this invention is that they are susceptible under appropriate circumstances to pathogenic agents that are tropic for primate liver cells. Such agents include hepatitis A, B, C, and delta, Epstein-Barr virus (EBV), cytomegalovirus (CMV), tuberculosis, and malaria. For example, infectivity by hepatitis B can be determined by combining cultured forward programming-derived hepatocytes with a source of infectious hepatitis B particles (such as serum from a human HBV carrier). The liver cells can then be tested for synthesis of viral core antigen (HBcAg) by immunohistochemistry or real time PCR.

The skilled reader will readily appreciate that an advantage of forward programming-derived hepatocytes is that they will be essentially free of other cell types that typically contaminate primary hepatocyte cultures isolated from adult or fetal liver tissue. Markers characteristic of sinusoidal endothelial cells include Von Willebrand factor, CD4, CD14, and CD32. Markers characteristic of bile duct epithelial cells include cytokeratin-7, cytokeratin-19, and γ-glutamyl transpeptidase. Markers characteristic of stellate cells include α-smooth muscle actin (α-SMA), vimentin, synaptophysin, glial fibrillary acidic protein (GFAP), neural-cell adhesion molecule (N-CAM), and presence of lipid droplets (detectable by autofluorescence or staining by oil red O). Markers characteristic of Kupffer cells include CD68, certain lectins, and markers for cells of the macrophage lineage (such as HLA Class II, and mediators of phagocytosis). Forward programming-derived hepatocytes can be characterized as essentially free of some or all of these cell types if less than 0.1% (preferably less than 100 or 10 ppm) bear markers or other features of the undesired cell type, as determined by immunostaining and fluorescence-activated quantitation, or other appropriate technique.

Hepatocytes provided by forward programming according to certain aspects of this invention can have a number of the features of the stage of cell they are intended to represent. The more of these features that are present in a particular cell, the more it can be characterized as a cell of the hepatocyte lineage. Cells having at least 2, 3, 5, 7, or 9 of these features are increasingly more preferred. In reference to a particular cell population as may be present in a culture vessel or a preparation for administration, uniformity between cells in the expression of these features is often advantageous. In this circumstance, populations in which at least about 40%, 60%, 80%, 90%, 95%, or 98% of the cells have the desired features are increasingly more preferred.

Other desirable features of hepatocytes provided in certain aspects of this invention are an ability to act as target cells in drug screening assays, and an ability to reconstitute liver function, both in vivo, and as part of an extracorporeal device. These features are described further in sections that follow.

### VII. USE OF HEPATOCYTES

The hepatocytes provided by methods and compositions of certain aspects of the invention can be used in a variety of applications. These include but not limited to transplantation or implantation of the hepatocytes in vivo; screening cytotoxic compounds, carcinogens, mutagens growth/regulatory factors, pharmaceutical compounds, etc., in vitro; elucidating the mechanism of liver diseases and infections; studying the mechanism by which drugs and/or growth factors operate; diagnosing and monitoring cancer in a patient; gene therapy; and the production of biologically active products, to name but a few.

A. Test Compound Screening

Forward programming-derived hepatocytes of this invention can be used to screen for factors (such as solvents, small molecule drugs, peptides, and polynucleotides) or environmental conditions (such as culture conditions or manipulation) that affect the characteristics of hepatocytes provided herein.

In some applications, stem cells (differentiated or undifferentiated) are used to screen factors that promote maturation of cells along the hepatocyte lineage, or promote proliferation and maintenance of such cells in long-term culture. For example, candidate hepatocyte maturation factors or growth factors are tested by adding them to stem cells in different wells, and then determining any phenotypic change that results, according to desirable criteria for further culture and use of the cells.

Particular screening applications of this invention relate to the testing of pharmaceutical compounds in drug research. The reader is referred generally to the standard textbook In vitro Methods in Pharmaceutical Research, Academic Press, 1997, and U.S. Pat. No. 5,030,015). In certain aspects of this invention, cell programmed to the hepatocyte lineage play the role of test cells for standard drug screening and toxicity assays, as have been previously performed on hepatocyte cell lines or primary hepatocytes in short-term culture. Assessment of the activity of candidate pharmaceutical compounds generally involves combining the hepatocytes provided in certain aspects of this invention with the candidate compound, determining any change in the morphology, marker phenotype, or metabolic activity of the cells that is attributable to the compound (compared with untreated cells or cells treated with an inert compound), and then correlating the effect of the compound with the observed change. The screening may be done either because the compound is designed to have a pharmacological effect on liver cells, or because a compound designed to have effects elsewhere may have unintended hepatic side effects. Two or more drugs can be tested in combination (by combining with the cells either simultaneously or sequentially), to detect possible drug-drug interaction effects.

In some applications, compounds are screened initially for potential hepatotoxicity (Castell et al., 1997). Cytotoxicity can be determined in the first instance by the effect on cell viability, survival, morphology, and leakage of enzymes into the culture medium. More detailed analysis is conducted to determine whether compounds affect cell function (such as gluconeogenesis, ureogenesis, and plasma protein synthesis) without causing toxicity. Lactate dehydrogenase (LDH) is a good marker because the hepatic isoenzyme (type V) is stable in culture conditions, allowing reproducible measurements in culture supernatants after 12-24 h incubation. Leakage of enzymes such as mitochondrial glutamate oxaloacetate transaminase and glutamate pyruvate transaminase can also be used. Gomez-Lechon et al. (1996) describes a microassay for measuring glycogen, which can be used to measure the effect of pharmaceutical compounds on hepatocyte gluconeogenesis.

Other current methods to evaluate hepatotoxicity include determination of the synthesis and secretion of albumin, cholesterol, and lipoproteins; transport of conjugated bile acids and bilirubin; ureagenesis; cytochrome p450 levels and activities; glutathione levels; release of α-glutathione s-transferase; ATP, ADP, and AMP metabolism; intracellular K+ and Ca2+ concentrations; the release of nuclear matrix proteins or oligonucleosomes; and induction of apoptosis (indicated by cell rounding, condensation of chromatin, and nuclear fragmentation). DNA synthesis can be measured as [³H]-thymidine or BrdU incorporation. Effects of a drug on DNA synthesis or structure can be determined by measuring DNA synthesis or repair. [³H]-thymidine or BrdU incorporation, especially at unscheduled times in the cell cycle, or above the level required for cell replication, is consistent with a drug effect. Unwanted effects can also include unusual rates of sister chromatid exchange, determined by metaphase spread. The reader is referred to Vickers (1997) for further elaboration.

B. Liver Therapy and Transplantation

This invention also provides for the use of hepatocytes provided herein to restore a degree of liver function to a subject needing such therapy, perhaps due to an acute, chronic, or inherited impairment of liver function.

To determine the suitability of hepatocytes provided herein for therapeutic applications, the cells can first be tested in a suitable animal model. At one level, cells are assessed for their ability to survive and maintain their phenotype in vivo. Hepatocytes provided herein are administered to immunodeficient animals (such as SCID mice, or animals rendered immunodeficient chemically or by irradiation) at a site amenable for further observation, such as under the kidney capsule, into the spleen, or into a liver lobule. Tissues are harvested after a period of a few days to several weeks or more, and assessed as to whether starting cell typess such as pluripotent stem cells are still present. This can be performed by providing the administered cells with a detectable label (such as green fluorescent protein, or β-galactosidase); or by measuring a constitutive marker specific for the administered cells. Where hepatocytes provided herein are being tested in a rodent model, the presence and phenotype of the administered cells can be assessed by immunohistochemistry or ELISA using human-specific antibody, or by RT-PCR analysis using primers and hybridization conditions that cause amplification to be specific for human polynucleotide sequences. Suitable markers for assessing gene expression at the mRNA or protein level are provided in elsewhere in this disclosure. General descriptions for determining the fate of hepatocyte-like cells in animal models is provided in Grompe et al. (1999); Peeters et al. (1997); and Ohashi et al. (2000).

At another level, hepatocytes provided herein are assessed for their ability to restore liver function in an animal lacking full liver function. Braun et al. (2000) outline a model for toxin-induced liver disease in mice transgenic for the HSV-tk gene. Rhim et al. (1995) and Lieber et al. (1995) outline models for liver disease by expression of urokinase. Mignon et al. (1998) outline liver disease induced by antibody to the cell-surface marker Fas. Overturf et al. (1998) have developed a model for Hereditary Tyrosinemia Type I in mice by targeted disruption of the Fah gene. The animals can be rescued from the deficiency by providing a supply of 2-(2-nitro-4-fluoro-methyl-benzyol)-1,3-cyclohexanedione (NTBC), but they develop liver disease when NTBC is withdrawn. Acute liver disease can be modeled by 90% hepatectomy (Kobayashi et al., 2000). Acute liver disease can also be modeled by treating animals with a hepatotoxin such as galactosamine, CCl4, or thioacetamide.

Chronic liver diseases, such as cirrhosis, can be modeled by treating animals with a sub-lethal dose of a hepatotoxin long enough to induce fibrosis (Rudolph et al., 2000). Assessing the ability of hepatocytes provided herein to reconstitute liver function involves administering the cells to such animals, and then determining survival over a 1 to 8 week period or more, while monitoring the animals for progress of the condition. Effects on hepatic function can be determined by evaluating markers expressed in liver tissue, cytochrome p450 activity, and blood indicators, such as alkaline phosphatase activity, bilirubin conjugation, and prothrombin time), and survival of the host. Any improvement in survival, disease progression, or maintenance of hepatic function according to any of these criteria relates to effectiveness of the therapy, and can lead to further optimization.

Hepatocytes provided in certain aspects of this invention that demonstrate desirable functional characteristics according to their profile of metabolic enzymes, or efficacy in animal models, may also be suitable for direct administration to human subjects with impaired liver function. For purposes of hemostasis, the cells can be administered at any site that has adequate access to the circulation, typically within the abdominal cavity. For some metabolic and detoxification functions, it is advantageous for the cells to have access to the biliary tract. Accordingly, the cells are administered near the liver (e.g., in the treatment of chronic liver disease) or the spleen (e.g., in the treatment of fulminant hepatic failure). In one method, the cells administered into the hepatic circulation either through the hepatic artery, or through the portal vein, by infusion through an in-dwelling catheter. A catheter in the portal vein can be manipulated so that the cells flow principally into the spleen, or the liver, or a combination of both. In another method, the cells are administered by placing a bolus in a cavity near the target organ, typically in an excipient or matrix that will keep the bolus in place. In another method, the cells are injected directly into a lobe of the liver or the spleen.

The hepatocytes provided in certain aspects of this invention can be used for therapy of any subject in need of having hepatic function restored or supplemented. Human conditions that may be appropriate for such therapy include fulminant hepatic failure due to any cause, viral hepatitis, drug-induced liver injury, cirrhosis, inherited hepatic insufficiency (such as Wilson's disease, Gilbert's syndrome, or α1-antitrypsin deficiency), hepatobiliary carcinoma, autoimmune liver disease (such as autoimmune chronic hepatitis or primary biliary cirrhosis), and any other condition that results in impaired hepatic function. For human therapy, the dose is generally between about 10⁹ and 10¹² cells, and typically between about 5×10⁹ and 5×10¹⁰ cells, making adjustments for the body weight of the subject, nature and severity of the affliction, and the replicative capacity of the administered cells. The ultimate responsibility for determining the mode of treatment and the appropriate dose lies with the managing clinician.

C. Use in a Liver Assist Device

Certain aspects of this invention include hepatocytes provided herein that are encapsulated or part of a bioartificial liver device. Various forms of encapsulation are described in Cell Encapsulation Technology and Therapeutics, 1999. Hepatocytes provided in certain aspects of this invention can be encapsulated according to such methods for use either in vitro or in vivo.

Bioartificial organs for clinical use are designed to support an individual with impaired liver function—either as a part of long-term therapy, or to bridge the time between a fulminant hepatic failure and hepatic reconstitution or liver transplant. Bioartificial liver devices are reviewed by Macdonald et al. (1999) and exemplified in U.S. Pat. Nos. 5,290,684, 5,624,840, 5,837,234, 5,853,717, and 5,935,849. Suspension-type bioartificial livers comprise cells suspended in plate dialysers, microencapsulated in a suitable substrate, or attached to microcarrier beads coated with extracellular matrix. Alternatively, hepatocytes can be placed on a solid support in a packed bed, in a multiplate flat bed, on a microchannel screen, or surrounding hollow fiber capillaries. The device has an inlet and outlet through which the subject's blood is passed, and sometimes a separate set of ports for supplying nutrients to the cells.

Hepatocytes are prepared according to the methods described earlier, and then plated into the device on a suitable substrate, such as a matrix of Matrigel® or collagen. The efficacy of the device can be assessed by comparing the composition of blood in the afferent channel with that in the efferent channel—in terms of metabolites removed from the afferent flow, and newly synthesized proteins in the efferent flow.

Devices of this kind can be used to detoxify a fluid such as blood, wherein the fluid comes into contact with the hepatocytes provided in certain aspects of this invention under conditions that permit the cell to remove or modify a toxin in the fluid. The detoxification will involve removing or altering at least one ligand, metabolite, or other compound (either natural or synthetic) that is usually processed by the liver. Such compounds include but are not limited to bilirubin, bile acids, urea, heme, lipoprotein, carbohydrates, transferrin, hemopexin, asialoglycoproteins, hormones like insulin and glucagon, and a variety of small molecule drugs. The device can also be used to enrich the efferent fluid with synthesized proteins such as albumin, acute phase reactants, and unloaded carrier proteins. The device can be optimized so that a variety of these functions is performed, thereby restoring as many hepatic functions as are needed. In the context of therapeutic care, the device processes blood flowing from a patient in hepatocyte failure, and then the blood is returned to the patient.

D. Distribution for Commercial, Therapeutic, and Research Purposes

For purposes of manufacture, distribution, and use, the hepatocyte lineage cells of this invention are typically supplied in the form of a cell culture or suspension in an isotonic excipient or culture medium, optionally frozen to facilitate transportation or storage.

This invention also includes different reagent systems, comprising a set or combination of cells that exist at any time during manufacture, distribution, or use. The cell sets comprise any combination of two or more cell populations described in this disclosure, exemplified but not limited to programming-derived cells (hepatocyte lineage cells, their precursors and subtypes), in combination with undifferentiated stem cells, somatic cell-derived hepatocytes, or other differentiated cell types. The cell populations in the set sometimes share the same genome or a genetically modified form thereof. Each cell type in the set may be packaged together, or in separate containers in the same facility, or at different locations, at the same or different times, under control of the same entity or different entities sharing a business relationship.

### VIII. CELLS AND METHODS FOR TESTING CANDIDATE GENES IN FORWARD PROGRAMMING

The ability of a particular candidate gene or a combination of candidate genes to act as forward programming factors for a specific cell type, such as hepatocytes, can be tested using the methods and cells provided in this disclosure. Efficacy of particular candidate genes or combinations of candidate genes in forward programming can be assessed by their effect on cell morphology, marker expression, enzymatic activity, proliferative capacity, or other features of interest, which is then determined in comparison with parallel cultures that did not include the candidate genes or combinations. Candidate genes may be transcription factors important for differentiation into desired cell types or for function of the desired cell types.

In certain embodiments, starting cells, such as pluripotent stem cells, comprising at least one expression cassette for expression of a candidate gene or a combination of candidate genes may be provided. The expression cassette may comprise an externally controllable transcriptional regulatory element, such as an inducible promoter. The activity of these promoters may be induced by the presence or absence of biotic or abiotic factors. Inducible promoters are a very powerful tool in genetic engineering because the expression of genes operably linked to them can be turned on or off at certain stages of development of an organism or in a particular tissue. Tet-On and Tet-Off inducible gene expression systems based on the essential regulatory components of the E. coli tetracycline-resistance operon may be used. Once established in the starting cells, the inducer doxycycline (Dox, a tetracycline derivative) could control the expression system in a dose-dependent manner, allowing to precisely modulate the expression levels of candidate genes.

To aid identification of desired cell types, the starting cells may further comprise a cell-specific or tissue-specific reporter expression cassette. The reporter expression cassette may comprise a reporter gene operably linked to a transcriptional regulatory element specific for the desired cell types. For example, the reporter expression cassette may comprise a hepatocyte-specific promoter for hepatocyte production, isolation, selection, or enrichment. The reporter gene may be any selectable or screenable marker gene known in the art and exemplified in the preceding disclosure.

### IX. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Forward Programming of Hepatocytes Via Genetic and Chemical Means

Alternative approaches for hepatocyte differentiation from human ESC/iPSCs are shown in FIG. 1. Hepatic lineage cells, such as mature hepatocytes, can be efficiently induced from human ESC/iPSCs via expression of an appropriate transgene combination (top box), bypassing most, if not all, developmental stages required during normal differentiation (bottom box).

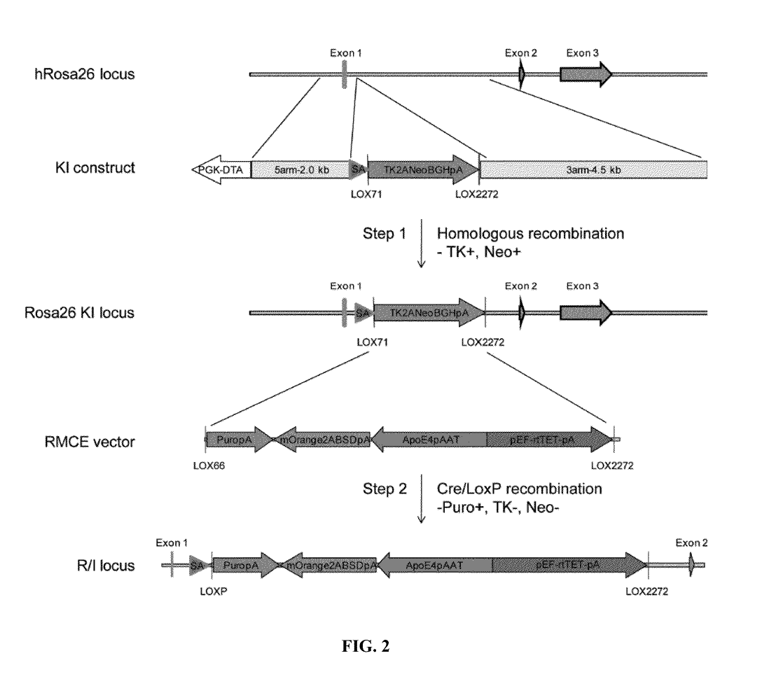

Human ESC/iPSC reporter/inducible (R/I) lines were established for hepatocyte differentiation (FIG. 2). The human Rosa26 locus on chromosome 3 was selected to allow the expression of both hepatocyte-specific reporter and rtTET, while minimizing the chromosome location-dependent silencing effect. First, the LoxP recombination sites (LOX71 and LOX2272) were introduced into a site between exon 1 and exon 2 of human ROSA 26 gene via homologous recombination. The targeting construct (KI construct) used the phosphoglycerate kinase promoter (PGK)-driven expression of diphtheria toxin A fragment gene (DTA) for negative selection, and contains a ˜2.0 kb 5′ arm and a 4.5 kb 3′ arm. A splicing acceptor signal from human BCL2 gene (SA) was placed in front of LOX71 site to allow the expression of selection markers from the endogenous human ROSA26 promoter. The coding region for thymidine kinase (TK) was included to enable negative selection against incorrect Cre/LoxP recombination events at step 2 using ganciclovir. The neomycin phosphotransferase (Neo) was used for positive selection during homologous recombination (step 1). The foot-and-mouth disease virus peptide (F2A) was used to co-express the TK and Neo genes from the endogenous human ROSA26 promoter. BGHpA is a polyadenylation signal derived from bovine growth hormone gene. The homologous recombination yielded parental human ESC/iPSC lines for efficient cassette exchange via Cre/LoxP recombination. To establish reporter/inducible cell lines for hepatocyte differentiation, F2A peptide linked marker gene mOrange and Blasticidin S deaminase (BSD) (driven by a hepatocyte-specific promoter ApoE4pAAT) and rtTET (driven by the constitutively active eukaryotic elongation factor 1α promoter—pEF) was introduced into the Rosa 26 locus by lipid-mediated cotransfection of the recombination mediated cassette exchange (RMCE) vector and a Cre-expressing plasmid. The puromycin N-acetyl-transferase (Puro) was used to select for recombination events. The correctly recombined R/I cells are resistant to puromycin (Puro⁺) and ganciclovir (TK⁻), and sensitive to geneticin selection (Neo).

The Tet-On inducible gene expression was confirmed in human H1 ESC R/I lines (FIGS. 3A-3C). The EGFP driven by the Ptight promoter (an rtTET-responsive inducible promoter) was introduced into human ESC R/I lines using Fugene HD-mediated transfection of both vectors in FIG. 3A. Human ESCs with stable PiggyBac transposon integration were selected with geneticin (100 μg/ml). Images are shown in FIG. 3B with human ESC R/I lines after 2 days induction with or without Doxycycline (1 μg/ml). EGFP expression was analyzed by flow cytometry in human ESC R/I lines after 4 days induction with or without Doxycycline (1 μg/ml) (FIG. 3C). After 4 days of Doxycycline induction, 83.3% human ESC R/I lines showed stable PiggyBac transposon integration by EGFP expression.

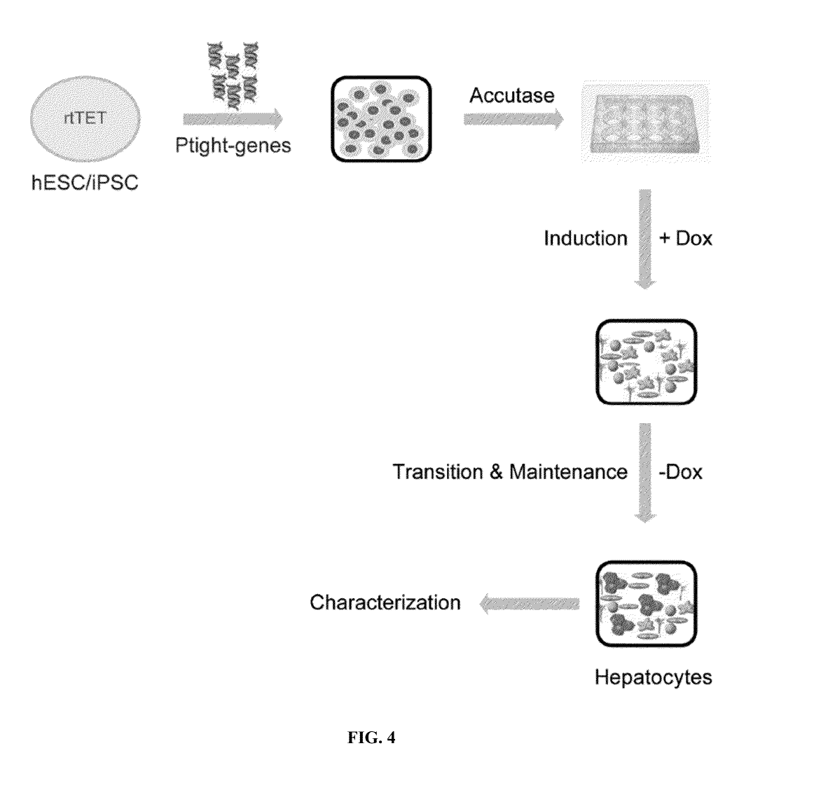

A diagram illustrating hepatocyte forward programming from human ESCs/iPSCs is shown in FIG. 4. Genes that are either implicated in hepatic differentiation during normal mammalian development or enriched in adult hepatocytes were cloned into the PiggyBac vector (FIG. 3) under the control of the Ptight promoter (Table 1). To find transcription factors that are able to directly impose mature hepatic fate upon human ESCs, various combinations of transgene-expressing PiggyBac vectors along with the hPBase-expressing vector were introduced into the human ESCs having constitutive expression of rtTET through nucleofection (Minis Ingenio Electroporation solution: cat#MIR50114; program: Amaxa B-016). Nucleofected human ESCs were cultured on matrigel in mTeSR1 (Stem Cell Technologies). Following geneticin (100 μg/ml) selection for stable genomic transgene integration (cells were passaged at lease once prior to differentiation), human ESCs were individualized by accutase treatment and plated to matrigel-coated 12-well plates. Doxycycline (1 μg/ml) was added the next day to induce transgene expression in Hepatocyte Maintenance Medium (HMM, Lonza) supplemented with 0.5 μg/ml insulin, 0.1 μM dexamethasone (dex), and 50 ng/ml Oncostatin M (OSM). After transgene induction for the appropriate number of days, doxycycline was removed, and cells were allowed to transition to hepatocyte-like cells and were maintained in HMM supplemented with 0.5 μg/ml insulin, 0.1 μM dex, and 50 ng/ml OSM prior to characterization. Where appropriate, small molecules, such as MEK inhibitor PD0325901, TGFβ kinase/activin receptor like kinase (ALK5) inhibitor A 83-01, and an analogue of the natural signaling molecule cyclic AMP 8-Bromoadenosine 3′,5′-cyclic monophosphate (8-Br-cAMP), were added during hepatic programming.

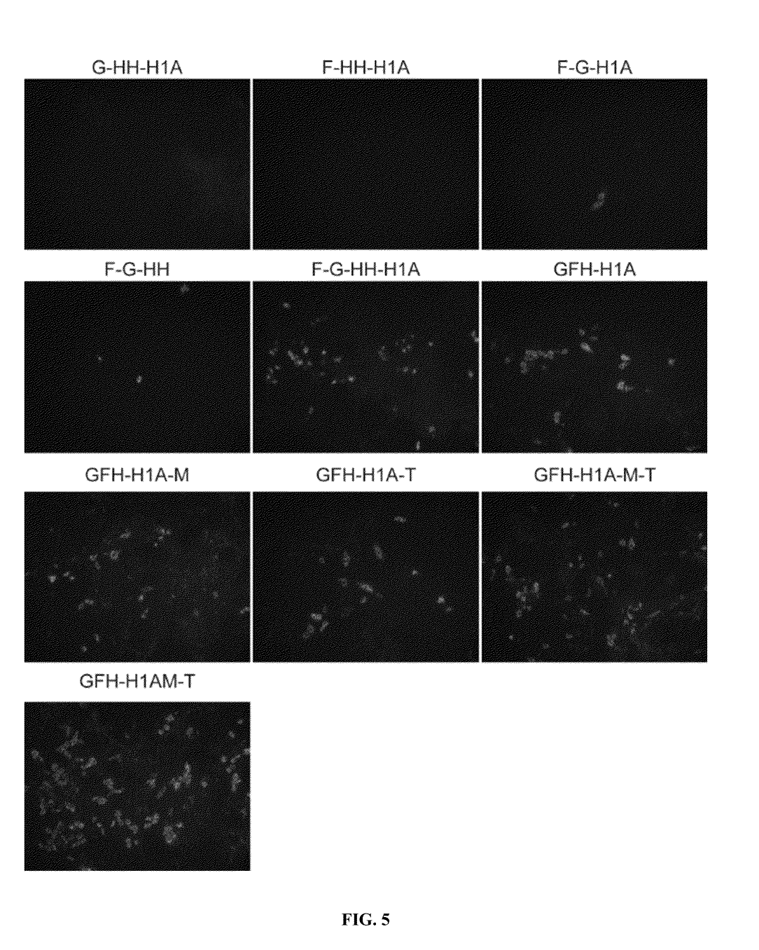

Human rtTET-expressing ESCs were transfected with various combinations of transgenes and/or co-expression vectors. Following drug selection for stable transgene integration, cells were individualized with accutase, and plated to matrigel-coated 12-well plates at about 0.2×10⁶ cells/well in mTeSR supplemented with 10 μM HA100 to facilitate cell attachment (day 0). From day 1 to day 7 post-plating, transgene expression was induced with 1 μg/ml doxycycline in HMM supplemented with 0.5 μg/ml insulin, 0.1 μM dex, and 50 ng/ml OSM. From day 7 on, cells were maintained in HMM supplemented with 0.5 μg/ml insulin, 0.1 μM dex, and 50 ng/ml OSM. Culture medium was replaced every other day during programming. On day 13, programming cultures were stained with mouse-anti-human albumin monoclonal antibody (1:5000, Cedarlane, Cat# CL2513A) followed by Alexa Fluor 488 donkey-anti-mouse IgG (H+L) secondary antibody (1:1000, Invitrogen, Cat# A-21202). Among the transgenes and coexpression vectors tested, FOXA2, GATA4, HHEX and HNF1A appeared to be required for successful hepatic reprogramming, while MAFB and TBX3 affected efficiency (FIG. 5). Improved hepatic programming efficiency was observed with GFH and H1AM coexpression vectors as defined in the description of FIG. 5.

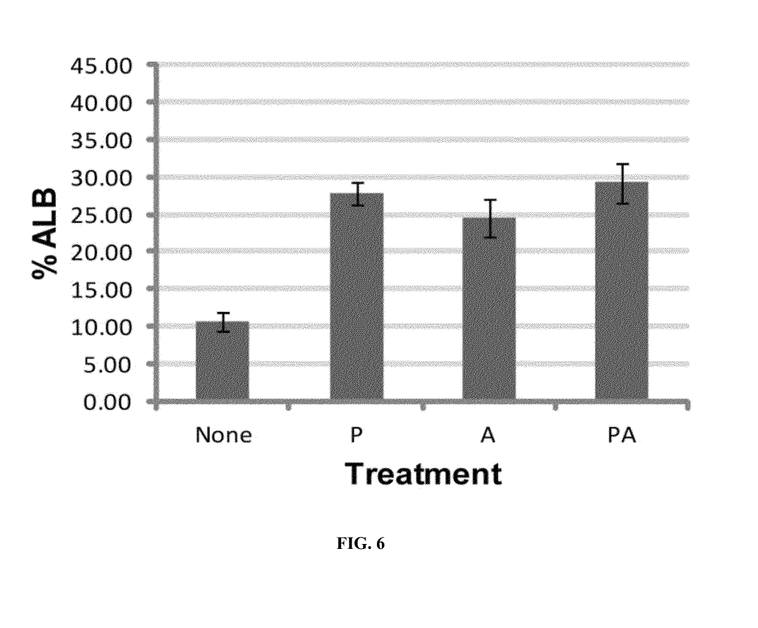

To determine the effect of MEK inhibitor PD0325901 (P) and TGFβ kinase/activin receptor like kinase (ALK5) inhibitor A 83-01 (A) on hepatic programming efficiency, human rtTET-expressing ESCs transfected with GFH, H1AM and TBX3 were plated on matrigel-coated 12-well plates at about 0.2×10⁶ cells/well in mTeSR supplemented with 10 μM HA100 on day 0. PD0325901 (0.5 μM), A 83-01 (0.5 μM) or both were added along with doxycycline between day 1 and day 7 post-plating. Cells were collected for albumin (ALB) flow analysis on day 13 post-plating. As shown in the graph, the addition of P or A alone significantly improves % ALB-expressing cells (FIG. 6). Although P and A did not appear to have significant additive effect, both were included in the hepatic induction stage to ensure consistent hepatic programming from different human ESC/iPSC lines.

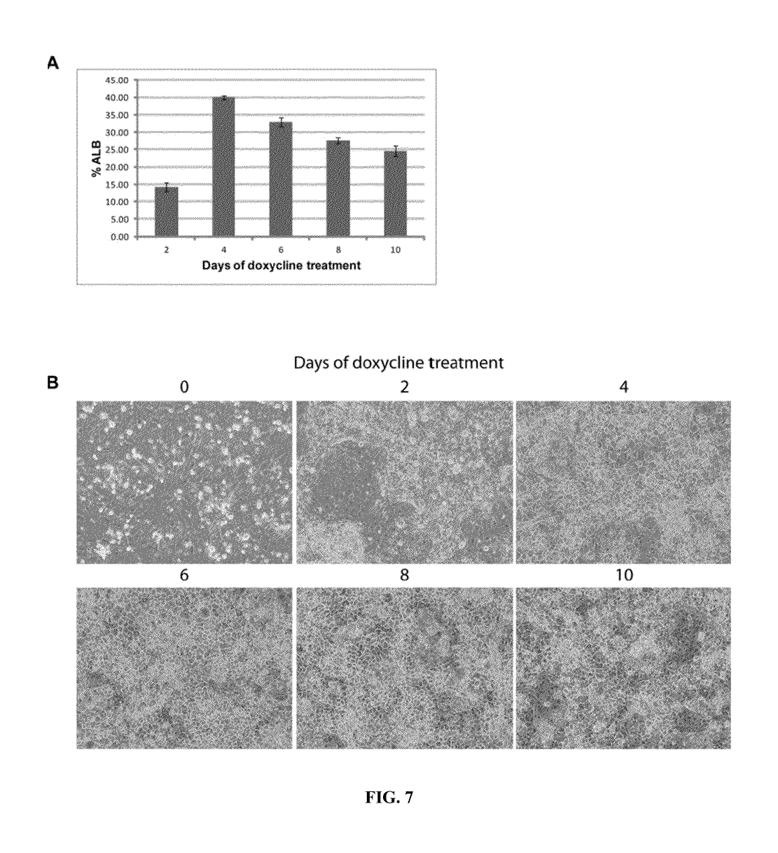

The effect of doxycycline induction duration on hepatic programming was determined by transfecting human rtTET-expressing ESCs with GFH, H1AM and TBX3. Transfected cells were plated on matrigel-coated 12-well plates at about 0.2×10⁶ cells/well in mTeSR supplemented with 10 μM HA100 on day 0. Doxycycline (1 μg/ml), P and A were added for 0, 2, 4, 6, 8, or 10 days. Cells were collected for ALB flow analysis on day 12 post-plating. As shown in FIG. 7A, there appeared to be an optimal time window for transgene induction (4 days of doxycycline treatment) for hepatic programming. In the absence of transgene expression, no hepatocyte-like cells were observed as shown in FIG. 7B, demonstrating the necessity of hepatic programming genes. With transgene expression, hepatocyte-like cells with polygonal shapes, distinct nuclei, and tight cell-cell contacts were readily observed.

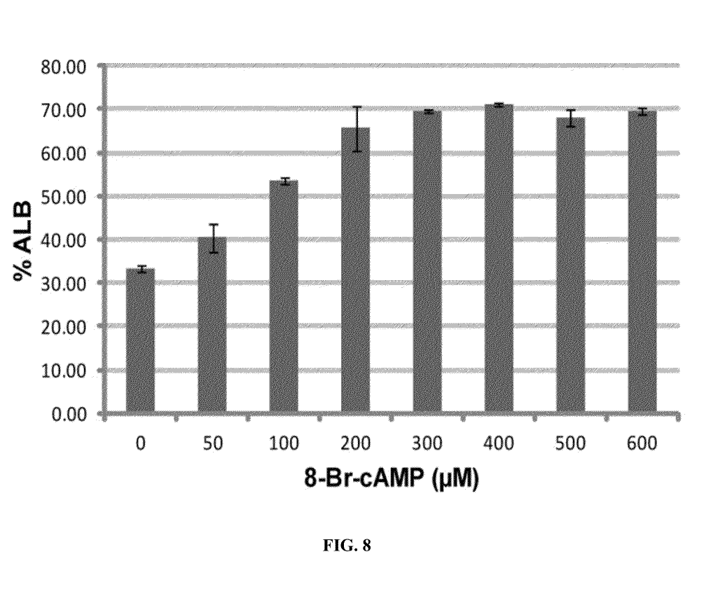

To determine the effect of cyclic AMP analog 8-Br-cAMP on hepatic programming, human rtTET-expressing ESCs transfected with GFH, H1AM and TBX3 were plated on matrigel-coated 12-well plates at about 0.2×10⁶ cells/well in mTeSR supplemented with 10 μM HA100 on day 0. Doxycycline (1 μg/ml), P and A were added between day 1 and day 7 post-plating. Following the removal of doxycycline, P and A on day 7, different concentrations of 8-Br-cAMP were added to promote hepatic transition. Cells were collected for ALB flow analysis on day 13 post-plating. As shown in the graph, the addition of 8-Br-cAMP significantly improved hepatic programming with a saturation concentration close to 200 μM (FIG. 8).

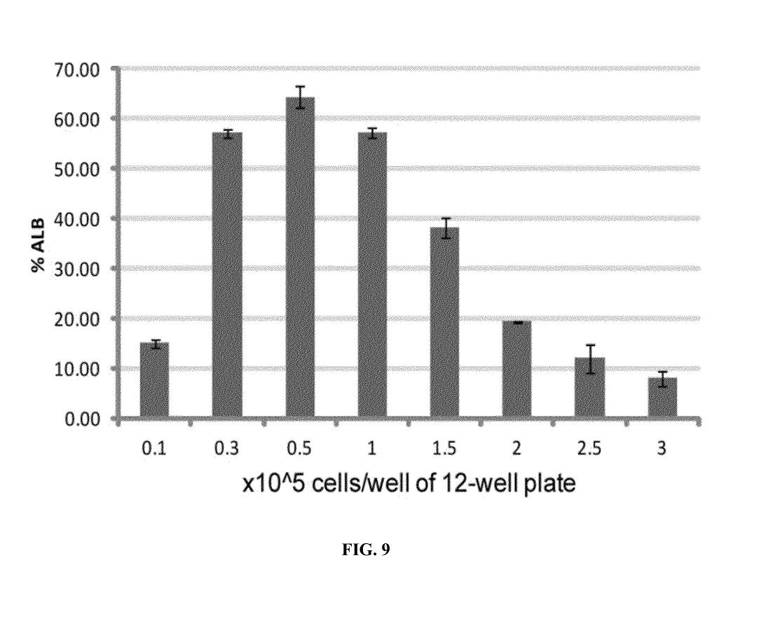

The effect of initial plating cell density on hepatic programming was determined by transfecting human rtTET-expressing ESCs with GFH, H1AM and TBX3. Transfected cells were plated on matrigel-coated 12-well plates at different numbers of cells/well in mTeSR supplemented with 10 μM HA100 on day 0. Doxycycline (1 μg/ml), P and A were added between day 1 and day 5 post-plating. Following the removal of doxycycline, P and A on day 5, 8-Br-cAMP (200 μM) was added to promote hepatic transition. Cells were collected for ALB flow analysis on day 11 post-plating. As shown in the graph, optimal hepatic programming required appropriate initial plating cell density (FIG. 9). Higher cell density culture, e.g., about 0.3×10⁶ cells/well, significantly reduced hepatic programming efficiency.

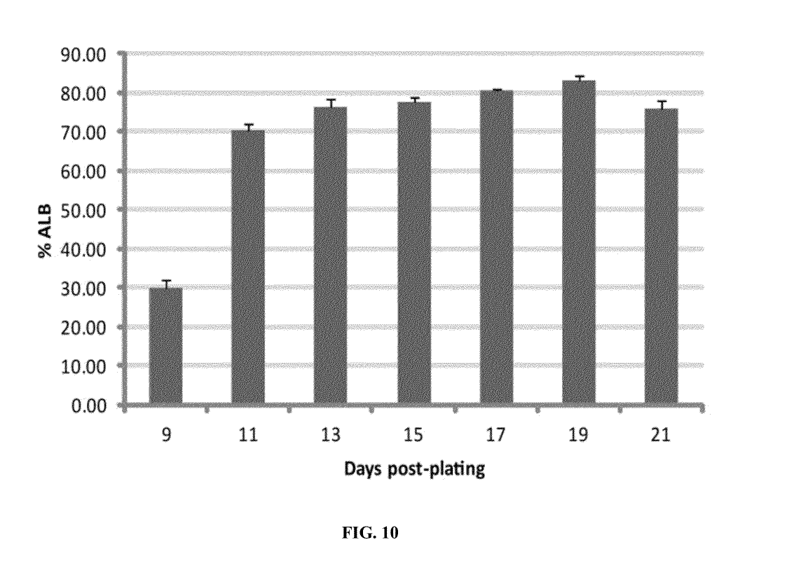

The kinetics of ALB expression during hepatic programming was determined by transfecting human rtTET-expressing ESCs with GFH, H1AM and TBX3. Transfected cells were plated on matrigel-coated 12-well plates at about 0.1×10⁶ cells/well in mTeSR supplemented with 10 μM HA100 on day 0. Doxycycline (1 μg/ml), P and A were added between day 1 and day 5 post-plating. Following the removal of doxycycline, P and A on day 5, 8-Br-cAMP (200 μM) was added to promote hepatic transition. Cells were collected for ALB flow analysis on different days post-plating as shown in the graph. As shown in the graph, the % ALB-expressing cells rapidly increase between day 9 and day 11 post-plating (FIG. 10). Following day 11, the % ALB-expressing cells remained constant. This suggested that the transition from non-hepatic cells to hepatocyte-like cells was complete at about day 11 post-plating with this protocol.

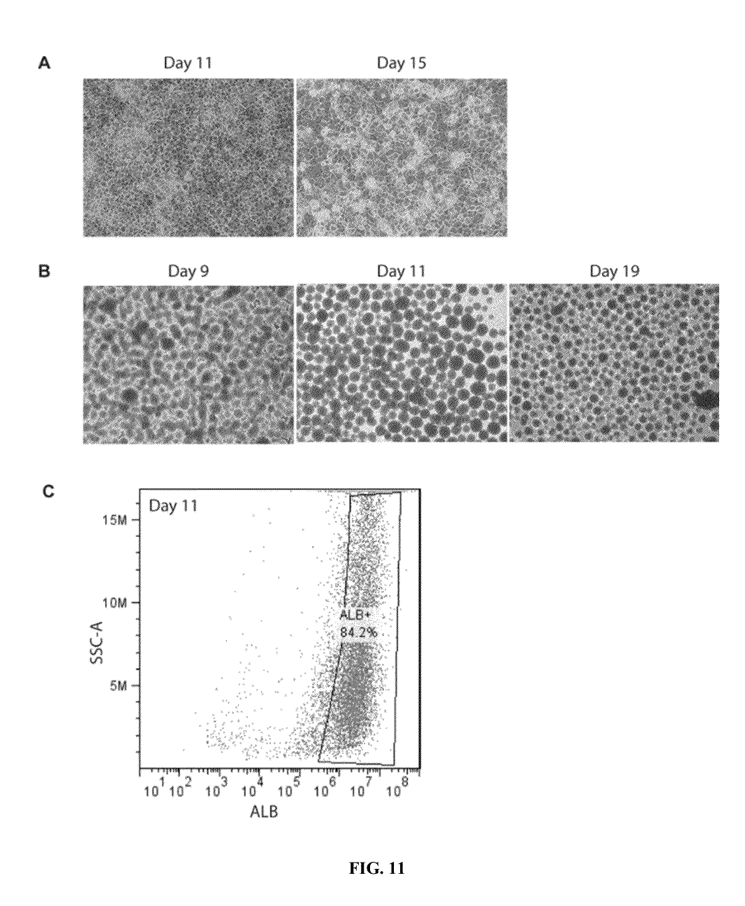

Inclusion of 3D culture facilitated hepatocyte survival and maturation. Programmed hepatocytes showed rapid deterioration in 2D culture (FIG. 11A). Specifically, the morphology of hepatocytes showed significant deterioration on day 15 after 4 days in HMM supplemented with insulin (0.5 μg/ml) and dexamethasone (0.1 μM), similar to primary human hepatocytes in 2D culture. When spheroids were formed at day 0, 3 and 5 of hepatic programming, it resulted in very poor yield at day 11 (input of hESCs: output of hepatocytes at day 11≈10:1). Spheroids were formed efficiently from day 7 of hepatic programming with reasonable yields (input of hESCs:output of hepatocytes at day 11≈1:1) (FIG. 11B). For hepatic programming, human rt-TET-expressing ESCs transfected with GFH, H1AM and TBX3 were plated onto matrigel-coated 6-well plates at ˜0.4×10⁶ cells/well in mTeSR supplemented with 10 μM HA100 on day 0. HMM supplemented with insulin (0.5 μg/ml), dexamethasone (0.1 μM), human leukemia inhibitory factor (hLIF: 5 ng/ml in place of OSM), doxycycline (1 μg/ml), P and/or A were added between day 1 and day 5 post-plating. Following the removal of doxycycline, P and/or A on day 5, HMM supplemented with insulin (0.5 μg/ml), dexamethasone (0.1 μM), hLIF (L, 5 ng/ml), 8-Br-cAMP (B, 200 μM) and sodium ascorbate (AA, 100 μg/ml) (HMM+LBAA) was added to promote hepatic transition. To prepare spheroids, day 7 hepatic programming cultures were washed once with 2 ml of 0.5 mM EDTA and 0.5 mM EGTA prepared in Ca²⁺ and Mg²⁺-free PBS per well of 6-well plates and dissociated with pre-warmed 1.5 ml per well of 0.05% Trypsin-EDTA (Invitrogen) supplemented with 0.5 mM EGTA for 6-7 minutes at 37° C. Following dissociation, HMM supplemented with 10% FBS was used to neutralize the trypsin. Cells were collected and washed once with HMM at 1200 rpm for 5 minutes. For spheroid formation, cells were resuspended in HMM+LBAA (˜6 ml for every 4 wells of the 6-well plates) and transferred to T25 flasks coated with 10% polyHema to prevent cell attachment (˜6 ml per flask). T25 flasks were placed on a rocker at 15 rpm in cell culture incubator. Spheroids were efficiently formed by day 9. To prevent spheroid clumping, ˜3 mg/ml of Albumax I or II (Invitrogen) was added to HMM+LBAA on day 9. Similar to 2D culture, the % ALB-positive cells nearly reached saturation in day 11 3D spheroids (FIG. 11C). After day 11, spheroids were maintained in HMM supplemented with insulin (0.5 μg/ml) and dexamethasone (0.1 μM) to promote further maturation (>31 days) with gradual shrinkage of spheroids (compare day 19 and day 11 spheroids) suggesting cell loss.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,683,202
U.S. Pat. No. 5,030,015
U.S. Pat. No. 5,290,684
U.S. Pat. No. 5,302,523
U.S. Pat. No. 5,322,783
U.S. Pat. No. 5,384,253
U.S. Pat. No. 5,460,964
U.S. Pat. No. 5,464,765
U.S. Pat. No. 5,486,359
U.S. Pat. No. 5,550,318
U.S. Pat. No. 5,525,625
U.S. Pat. No. 5,538,877
U.S. Pat. No. 5,538,880
U.S. Pat. No. 5,550,318
U.S. Pat. No. 5,563,055
U.S. Pat. No. 5,580,859
U.S. Pat. No. 5,589,466
U.S. Pat. No. 5,591,616
U.S. Pat. No. 5,610,042
U.S. Pat. No. 5,624,840
U.S. Pat. No. 5,635,387
U.S. Pat. No. 5,656,610
U.S. Pat. No. 5,677,136
U.S. Pat. No. 5,681,599
U.S. Pat. No. 5,702,932
U.S. Pat. No. 5,716,827
U.S. Pat. No. 5,736,396
U.S. Pat. No. 5,736,524
U.S. Pat. No. 5,750,397
U.S. Pat. No. 5,759,793
U.S. Pat. No. 5,780,448
U.S. Pat. No. 5,789,215
U.S. Pat. No. 5,811,094
U.S. Pat. No. 5,827,735
U.S. Pat. No. 5,827,740
U.S. Pat. No. 5,837,234
U.S. Pat. No. 5,837,539
U.S. Pat. No. 5,837,670
U.S. Pat. No. 5,843,780
U.S. Pat. No. 5,853,717
U.S. Pat. No. 5,908,782
U.S. Pat. No. 5,925,565
U.S. Pat. No. 5,928,906
U.S. Pat. No. 5,935,819
U.S. Pat. No. 5,935,849
U.S. Pat. No. 5,945,100
U.S. Pat. No. 5,981,274
U.S. Pat. No. 5,994,136
U.S. Pat. No. 5,994,624
U.S. Pat. No. 6,013,516
U.S. Pat. No. 6,458,589
U.S. Pat. No. 6,465,493
U.S. Pat. No. 6,506,574
U.S. Pat. No. 6,833,269
U.S. Pat. No. 6,906,089
U.S. Pat. No. 6,919,425
U.S. Pat. No. 6,991,897
U.S. Pat. No. 7,015,037
U.S. Pat. No. 7,399,632
U.S. Pat. No. 7,410,798
U.S. Pat. No. 7,410,773
U.S. Pat. No. 7,422,736
U.S. Pat. No. 7,442,548
U.S. application Ser. No. 13/546,365
U.S. Appln. Ser. 61/058,858
U.S. Appln. Ser. 61/172,079
U.S. Appln. Ser. 61/184,546
U.S. Pat. Publn. 2004/0039198
U.S. Pat. Publn. 2004/0063745
U.S. Pat. Publn. 2003/0166633
U.S. Pat. Publn. 2003/0082561
U.S. Pat. Publn. 2003/0040038
U.S. Pat. Publn. 2003/0211603
U.S. Pat. Publn. 2007/0116680
EP0412700
EP1507865
International Patent Publn. 2005/123902
PCT Publn. WO 94/09699
PCT Publn. WO 95/11308
PCT Publn. WO 95/06128
PCT Publn. WO 96/39487
PCT Publn. WO 99/20741
PCT Publn. WO 01/098482
PCT Publn. WO 01/081549
PCT Publn. WO 03/042405
PCT Publn. WO 05/028630
PCT Publn. WO 09/130,208
PCT Publn. WO 10/079,430
Alexander et al., Proc. Nat. Acad. Sci. U.S.A., 85:5092-5096, 1988.
Alison et al., Hepatol., 29:678-683, 1998.
Amit et al., Dev. Bio., 227:271-278, 2000.
Andrews et al., In: Teratocarcinomas and Embryonic Stem Cells, Robertson (Ed.), IRL Press,
207-246, 1987.
Aravind and Landsman, Nucleic Acids Res., 26(19):4413-4421, 1998.
Arnould et al., Protein Eng. Des. Sel., 24:27-31, 2011.
Asoh et al., Proc. Natl. Acad. Sci. U.S.A., 99(26):17107-17112, 2002.
Ausubel et al., In: Current Protocols in Molecular Biology, John, Wiley & Sons, Inc, New York, 1994.
Ausubel et al., Current Protocols in Molecular Biology, Greene Publ. Assoc. Inc. & John Wiley
& Sons, Inc., MA, 1996.
Bain et al., Biochem. J., 408(3):297-315, 2007.
Blomer et al., J. Virol., 71(9):6641-6649, 1997.
Boyer et al., Cell, 122(6):947-956, 2005.
Braun et al., Nature Med., 6:320, 2000.
Bublitz, Mol. Cell. Biochem., 108:141, 1991.
Buss et al., Mol. Cell. Biol., 8:3960-3963, 1988.
Byrne et al., Nature, 450(7169):497-502, 2007.
Capecchi, Nature, 348:109, 1990.
Cassiede et al., J. Bone Miner. Res., 11(9):1264-1273, 1996.
Castell et al., In: In vitro Methods in Pharmaceutical Research, Academic Press, 375-410, 1997.
Cell Encapsulation Technology and Therapeutics, Kuhtreiber et al. eds., Birkhauser, Boston Mass., 1999.
Chambers et al., Cell, 113(5):643-655, 2003.
Chen and Okayama, Mol. Cell Biol., 7(8):2745-2752, 1987.
Chesne et al., In: Liver Cells and Drugs, Guillouzo (Ed.), John Libbey Eurotext, London, 343-350, 1988.
Chevalier et al., Mol. Cell., 10:895-905, 2002.
Current Protocols in Stem Cell Biology, Bhatia et al. (Ed.), John Wiley and Sons, Inc., 2007.
Derossi et al., J. Biol. Chem., 269:10444-10450, 1994.
Derossi et al., J. Biol. Chem., 271:18188, 1996.
Derossi et al., Trends in Cell Biol., 8:84-87, 1998.
Durai et al., Nucleic Acids Res., 33:5978-5990, 2005.
Ehata et al., Cancer Sci., 98:127-133, 2007.
Ercolani et al., J. Biol. Chem., 263:15335-15341, 1988.
Evans et al., In: Cancer Principles and Practice of Oncology, Devita et al. (Eds.), Lippincot-Raven, N.Y., 1054-1087, 1997.
Fawell et al., Proc. Natl. Acad. Sci. U.S.A., 91:664-668, 1994.
Fechheimer et al., Proc. Natl. Acad. Sci. U.S.A., 84:8463-8467, 1987.
Fernandes et al., Nature Cell Biology, 6:1082-1093, 2004.
Ferry et al., Hum. Gene Ther., 9(14):1975-1981, 1998.
Follenzi et al., Hum. Gene Ther., 13(2):243-260, 2002.
Fraley et al., Proc. Natl. Acad. Sci. U.S.A., 76:3348-3352, 1979.
Frankel and Pabo, Cell, 55(6):1189-1193, 1988.
Gebhart and Wang, J. Cell Sci., 56233-56244, 1982.
Gehrke et al., Gene, 322:137-143, 2003.
Ghosh et al., J. Hepatol., 32(1Suppl):238-252, 2000.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Gomes-Lechon et al., In: In vitro Methods in Pharmaceutical Research, Academic Press, 129-153, 1997.
Gomez-Lechon et al., Anal. Biochem., 236:296, 1996.
Gopal, Mol. Cell Biol., 5:1188-1190, 1985.
Graham and Van Der Eb, Virology, 52:456-467, 1973.
Grompe et al., Sem. Liver Dis., 19:7, 1999.
Gronthos, Blood, 84(12):4164-4173, 1994.
Guillouzo, In: In vitro Methods in Pharmaceutical Research, Academic Press, pp 411-431, 1997.
Gump and Dowdy, Trends Mol. Med., 13:443-448, 2007.
Hancock et al., EMBO J., 10:4033-4039, 1991.
Harland and Weintraub, J. Cell Biol., 101(3):1094-1099, 1985.
Hill et al., Exp. Hematol., 24(8):936-943, 1996.
Ho et al., Cancer Res., 61(2):474-477, 2001.
Huang et al., Induction of functional hepatocyte-like cells from mouse fibroblasts by defined factors, Nature, 475:386-389, 2011.
Inman et al., Molec. Pharmacol., 62(1):65-74, 2002.
In vitro Methods in Pharmaceutical Research, Academic Press, 1997.
Jaiswal et al., J. Cell Biochem., 64(2):295-312, 1997.
Johnstone et al., Exp. Cell. Res., 238(1):265-272, 1998.
Kaeppler et al., Plant Cell Rep., 8:415-418, 1990.
Kaneda et al., Science, 243:375-378, 1989.
Karin et al., Cell, 36:371-379, 1989.
Kato et al., J. Biol. Chem., 266:3361-3364, 1991.
Keller et al., Curr. Opin. Cell Biol., 7(6):862-9, 1995.
Kennedy et al., Proc. Natl. Acad. Sci. U.S.A., 100:14269-14274, 2003.
Kilic et al., Stroke, 34:1304-1310, 2003.
Kim et al., Radiology, 255:75-82, 2010.
Kirchmaier and Sugden, J. Virol., 72(6):4657-4666, 1998.
Kirkeby et al., Biochem. Biophys. Meth., 24:225, 1992.
Klein et al., Nature, 327:70-73, 1987.
Kobayashi et al., Science, 287:1258, 2000.
Kramer et al., Mol. Ther., 7(3):375-385, 2003.
Langle-Rouault et al., J. Virol., 72(7):6181-6185, 1998.
Le et al., Blood, 89(4):1254-1259, 1997.
Leight and Sugden, Mol. Cell. Bio., 21:4149-4161, 2001.
Levitskaya et al., Proc. Natl. Acad. Sci. U.S.A., 94(23):12616-12621, 1997.
Lieber et al., Proc. Natl. Acad. Sci. U.S.A., 92:6210, 1995.
Lindgren et al., Trends in Pharmacol. Sci., 21:99-103, 2000.
Lindner et al., J. Virol., 82(12):5693-5702, 2008.
Macdonald et al., In: Cell Encapsulation Technology and Therapeutics, Kuhtreiber et al., eds., Birkhauser, Boston Mass., pp. 252-286, 1999.
Macejak and Sarnow, Nature, 353:90-94, 1991.
Mackey and Sugden, Mol. Cell. Biol., 19(5):3349-3359, 1999.
Makino et al., J. Clin. Invest., 103(5):697-705, 1999.
Maniatis et al., Molecular Cloning, A Laboratory Manual, Cold Spring Harbor Press, Cold Spring Harbor, N.Y., 1988.
Mann et al., Cell, 33:153-159, 1983.
Mann and Frankel, EMBO J., 10:1733-1739, 1991.
Manno et al., Nat. Med., 12(3):342-347, 2006.
Miao et al., Mol. Ther., 1(6):522-32, 2000.
Miao et al., Mol. Ther., 3:947-957, 2001.
Mignon et al., Nature Med., 4:1185, 1998.
Miller et al., Am. J. Clin. Oncol., 15(3):216-221, 1992.
Miller et al., Nat. Biotechnol., 29:143-148, 2011.
Miyoshi et al, J. Biomater. Sci. Polym. Ed., 9:227-237, 1998.
Nabel et al., Science, 244(4910):1342-1344, 1989.
Naldini et al., Science, 272(5259):263-267, 1996.
Nanbo et al., EMBO J., 26:4252-4262, 2007.
Ng, Nuc. Acid Res., 17:601-615, 1989.
Nicolas and Rubenstein, In: Vectors: A survey of molecular cloning vectors and their uses, Rodriguez and Denhardt, eds., Stoneham: Butterworth, pp. 494-513, 1988.
Nicolau et al., Methods Enzymol., 149:157-176, 1987.
Nicolau and Sene, Biochim. Biophys. Acta, 721:185-190, 1982.
Ockerman, Clin. Chim. Acta, 17:201, 1968.
Ohashi et al., Nature Med., 6:327, 2000.
Overturf et al., Human Gene Ther., 9:295, 1998.
Park et al., Eur. J. Cancer, 47:2642-2653, 2011.
Paskind et al., Virology, 67:242-248, 1975.
Passonneau and Lauderdale, Anal. Biochem., 60:405-415, 1974.
Peeters et al., Hepatology, 25:884, 1997.
Pelletier and Sonenberg, Nature, 334:320-325, 1988.
Pingoud and Silva, Nat. Biotechnol., 25:743-744, 2007.
Potrykus et al., Mol. Gen. Genet., 199(2):169-177, 1985.
Potten, Philos. Trans. R Soc. Lond. B Biol. Sci., 353:821-830, 1998.
Potter et al., Proc. Natl. Acad. Sci. U.S.A., 81:7161-7165, 1984.
Quitsche et al., J. Biol. Chem., 264:9539-9545, 1989.
Reubinoff et al., Nat. Biotechnol., 18:399-404, 2000.
Rhim et al., Proc. Natl. Acad. Sci. U.S.A., 92:4942, 1995.
Richards et al., Cell, 37:263-272, 1984.
Rippe et al., Mol. Cell. Biol., 10:689-695, 1990.
Rothbard et al., Nat. Med., 6(11):1253-1257, 2000.
Rudolph et al., Science, 287:1253, 2000.
Sambrook et al., In: Molecular Cloning: A Laboratory Manual, Vol. 1, Cold Spring Harbor Laboratory Press, Cold Spring Harbor, N.Y., (7) 7:19-17.29, 1989.
Sambrook and Russell, Molecular Cloning: A Laboratory Manual, 3rd Ed. Cold Spring Harbor Lab. Press, 2001.
Schwarze et al., Science, 285:1466-1467, 1999.
Schwarze et al., Science, 285:1569-1572, 1999.
Sears et al., J. Virol., 78(21):11487-11505, 2004.
Sekiya and Suzuki, Direct conversion of mouse fibroblasts to hepatocyte-like cells by defined factors, Nature, 475:390-393, 2011.
Sheehan and Hrapchak, In: Theory and Practise of Histotechnology, 2nd Ed., Battelle Memorial Institute, Columbus, Ohio, 1987.
Shen et al., DNA, 8(2):101-108, 1989.
Shiojiri, J. Embryol. Exp. Morph., 62:139, 1981.
Simonet et al., J. Biol. Chem., 268(11):8221-8229, 1993.
Smith, In: Origins and Properties of Mouse Embryonic Stem Cells, Annu. Rev. Cell. Dev. Biol., 2000.
Takahashi et al., Cell, 126:663-676, 2007.
Takahashi et al., Cell, 131:861-872, 2007.
Takahashi and Yamanaka, Cell, 126:663-676, 2006.
Tanaka et al., J. Immunol., 170(3):1291-1298, 2003.
Temin, In: Gene Transfer, Kucherlapati (Ed.), NY, Plenum Press, 149-188, 1986.
Thompson, In: Selected Histochemical and Histopathological Methods, Tomas (Ed.), Sprungfield, Ill., 1966.
Thomson et al., Proc. Natl. Acad. Sci. U.S.A., 92:7844-7848, 1995.
Thomson et al., Science, 282:1145, 1998.
Thomson and Marshall, Curr. Top. Dev. Biol., 38:133-165, 1998.
Thomson and Odorico, J. Trends. Biotechnol., 18:53-57, 2000.
Tojo et al., Cancer Sci., 96:791-800, 2005.
Tur-Kaspa et al., Mol. Cell. Biol., 6:716-718, 1986.
Uhl et al., Cancer Res., 64:7954-7961, 2004.
VandenDriessche et al., J. Thromb. Haemost., 5(1):16-24, 2007.
van der Laarse et al., Biotech. Histochem., 67:303, 1992.
Vickers, In: In vitro Methods in Pharmaceutical Research, Academic Press, 375-410, 1997
Watanabe et al., Nat. Neurosci., 8(3):288-96, 2005.
Watt, Philos. Trans. R. Soc. Lond. B. Biol. Sci., 353:831, 1997.
Wender et al., Proc. Natl. Acad. Sci. U.S.A., 97(24):13003-13008, 2000.
Wernig et al., Nature, 448(7151):318-324, 2007.
Wilson et al., Science, 244:1344-1346, 1989.
Wong et al., Gene, 10:87-94, 1980.
Wu and Wu, J. Biol. Chem., 262:4429-4432, 1987.
Wu and Wu, Biochemistry, 27: 887-892, 1988.
Xu et al., Nat. Biotechnol., 19:971-974, 2001.
Yakubov et al., Biochemical and Biophysical Research Communications, 394:189-193, 2010.
Yang and Russell, Proc. Natl. Acad. Sci. U.S.A., 87:4144-4148, 1990.
Yasmineh et al., Clin. Biochem., 25:109, 1992.
Ying et al., Cell, 115:281-292, 2003.
Yoo et al., J. Bone Joint Sure. Am., 80(12):1745-1757, 1998.
Yu et al., Science, 318:1917-1920, 2007.
Yu et al., Science, 324:797-801, 2009.
Yu and Thompson, Genes Dev., 22(15):1987-1997, 2008.
Yull et al., Transgenic Res., 4(1):70-74, 1995.
Zhang et al., Bioorganic Med. Chem. Letters, 10:2825-2828, 2000.
Zufferey et al., Nat. Biotechnol., 15(9):871-875, 1997.

1. A method of producing hepatocytes by forward programming of stem cells, comprising transfecting the stem cells with at least one exogenous expression cassette comprising the hepatocyte programming factor genes encoding FOXA2, GATA4, HHEX, HNF1A, and TBX3, thereby producing hepatocytes from forward programming of the stem cells.

2. The method of claim 1, wherein the at least one exogenous expression cassette is operably linked to an externally inducible transcriptional regulatory element.

3. The method of claim 1, further comprising contacting the stem cells with a MEK inhibitor and/or an ALK5 inhibitor.

4. The method of claim 3, wherein the MEK inhibitor is PD0325901.

5. The method of claim 3, wherein the ALK5 inhibitor is A 83-01.

6. The method of claim 3, further comprising contacting the stem cells with a cyclic AMP analog.

7. The method of claim 6, wherein the cyclic AMP analog is 8-Br-cAMP.

8. The method of claim 1, wherein the stem cells are mesenchymal stem cells, hematopoietic stem cells, embryonic stem cells, or induced pluripotent stem cells.

9. The method of claim 1, wherein the stem cells or progeny cells thereof further comprise a reporter expression cassette comprising a hepatocyte specific transcriptional regulatory element operably linked to a reporter gene.

10. The method of claim 9, wherein the hepatocyte-specific transcriptional regulatory element is a promoter of albumin, α-1-antitrypsin (AAT), cytochrome p450 3A4 (CYP3A4), apolipoprotein A-I, or APOE.

11. The method of claim 1, wherein the hepatocytes comprise one or more of the hepatocyte characteristics comprising:
(i) expression of one or more hepatocyte markers including glucose-6-phosphatase, albumin, α-1-antitrypsin (AAT), cytokeratin 8 (CK8), cytokeratin 18 (CK18), asialoglycoprotein receptor (ASGR), alcohol dehydrogenase 1, arginase Type I, cytochrome p450 3A4 (CYP3A4), liver-specific organic anion transporter (LST-1), or a combination thereof;
(ii) activity of glucose-6-phosphatase, CYP3A4, bile production or secretion, urea production, or xenobiotic detoxification;
(iii) hepatocyte morphological features; or
(iv) in vivo liver engraftment in an immunodeficient subject.

12. The method of claim 11, wherein the hepatocyte characteristic is albumin expression.

13. The method of claim 1, further comprising selecting or enriching for hepatocytes.

14. The method of claim 1, wherein the stem cells or progeny cells thereof are cultured in a medium comprising one or more growth factors including Oncostatin M (OSM).

15. The method of claim 1, comprising obtaining the hepatocytes less than or about 15 days after culturing in said conditions.

16. The method of claim 15, comprising obtaining the hepatocytes less than or about 10 days after culturing in said conditions.

17. A method of assessing a compound for a pharmacological or toxicological effect on a hepatocyte, comprising:
(a) contacting a hepatocyte provided by the method in accordance with claim 1 with the compound; and
(b) assaying a pharmacological or toxicological effect of the compound on the hepatocyte.

18. A hepatocyte or stem cells comprising:
(a) one or more exogenous expression cassettes comprising FOXA2, GATA4, HHEX, HNF1A, and TBX3; and
(b) a reporter expression cassette comprising a hepatocyte-specific promoter operably linked to a reporter gene.

19. A hepatocyte or stem cell comprising one or more exogenous expression cassettes, wherein the one or more exogenous expression cassettes comprise FOXA2, GATA4, HHEX, HNF1A, and TBX3, and at least one of the exogenous expression cassettes is operably linked to an externally inducible transcriptional regulatory element.

20. A cell population comprising hepatocytes, wherein at least 80% of the hepatocytes comprise one or more exogenous expression cassettes that comprises the genes encoding FOXA2, GATA4, HHEX, HNF1A, and TBX3.

21. A method of producing hepatocytes from stem cells comprising:
(a) transfecting the stem cells with at least one exogenous inducible expression cassette comprising at least the hepatocyte programming factor genes encoding FOXA2, GATA4, HHEX, HNF1A, and TBX3;
(b) inducing the expression of the at least one exogenous inducible expression cassette;
(c) contacting the stem cells with a MEK inhibitor and/or an ALK5 inhibitor; and
(d) contacting the stem cells with a cyclic AMP analog, thereby producing hepatocytes from stem cells.
